SEQUENCE LISTING (14 sequences)

SEQ ID NO: 1 - DNA, 19 bp, synthetic construct
  source 1..19: mol_type = other DNA
attcacgcgtgccatggag

SEQ ID NO: 2 - DNA, 20 bp, synthetic construct
  source 1..20: mol_type = other DNA
cgggtcgactcacgcatctt

SEQ ID NO: 3 - DNA, 26 bp, synthetic construct
  source 1..26: mol_type = other DNA
ttcacgcgtgccatggcctgtttgtg

SEQ ID NO: 4 - DNA, 38 bp, synthetic construct
  source 1..38: mol_type = other DNA
cgggtcgactcaagtcagcacaccggggttgacccaat

SEQ ID NO: 5 - DNA, 26 bp, synthetic construct
  source 1..26: mol_type = other DNA
ttcacgcgtgccatggcctgtttgtg

SEQ ID NO: 6 - DNA, 22 bp, synthetic construct
  source 1..22: mol_type = other DNA
cgggtcgactcatcaaaagggg

SEQ ID NO: 7 - protein, 21 aa, synthetic construct
MEVGWYRSPFSRVVHLYRNGK

SEQ ID NO: 8 - DNA, 744 bp, synthetic construct
  source 1..744: mol_type = other DNA
  misc_feature 472..528: note = transmembrane domain
  misc_feature 538..606: note = transmembrane domain
atggcctgtttgtggagcttctcttggcccagctgcttcctctcccttctcctcctccttctcctccagttgtcatgcagctatgcaggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgactctcatcgcacttgtgcctacgatcctcctgcaggtctctgtaggccttgtattcctcttcctgcagcacagactgagaggaaaacttcgtgcagaagtagagaatctccatcggacttttgatcctcacttcctgagggtgccctgctggaagataacactgtttgttattgtgcctgttcttggacccctggttgccttgatcatctgctacaactggctgcaccgaagactggcaggacagtttcttgaagagctaagaaaccccttttga

SEQ ID NO: 9 - protein, 247 aa, synthetic construct
  TRANSMEM 158..176
  TRANSMEM 180..202
MACLWSFSWPSCFLSLLLLLLLQLSCSYAGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLTLIALVPTILLQVSVGLVFLFLQHRLRGKLRAEVENLHRTFDPHFLRVPCWKITLFVIVPVLGPLVALIICYNWLHRRLAGQFLEELRNPF

SEQ ID NO: 10 - DNA, 474 bp, synthetic construct
  source 1..474: mol_type = other DNA
atggcctgtttgtggagcttctcttggcccagctgcttcctctcccttctcctcctccttctcctccagttgtcatgcagctatgcaggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtgctgacttga

SEQ ID NO: 11 - protein, 157 aa, synthetic construct
MACLWSFSWPSCFLSLLLLLLLQLSCSYAGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLT

SEQ ID NO: 12 - DNA, 510 bp, synthetic construct
  source 1..510: mol_type = other DNA
atggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatgcggccatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatgcgtga

SEQ ID NO: 13 - protein, 169 aa, synthetic construct
MEVGWYRSPFSRVVHLYRNGKDQDAEQAPEYRGRTELLKETISEGKVTLRIQNVRFSDEGGYTCFFRDHSYQEEAAMELKVEDAAMEVGWYRSPFSRVVHLYRNGKDQDAEQAPEYRGRTELLKETISEGKVTLRIQNVRFSDEGGYTCFFRDHSYQEEAAMELKVEDA

SEQ ID NO: 14 - protein, 29 aa, synthetic construct
MACLWSFSWPSCFLSLLLLLLLQLSCSYA

(19) United States
(12) Patent Application Publication
(10) Pub. No.: US 2026/0256843 A1
(43) Pub. Date: Sep. 3, 2026

(54) IMMUNE TOLERANCE INDUCTION FOR AUTO-IMMUNE DISEASES THROUGH PLATELET TARGETED GENE THERAPY INVOLVING MYELIN OLIGODENDROCYTE GLYCOPROTEIN (MOG) POLYPEPTIDE

(71) Applicants: The Medical College of Wisconsin, Inc., Milwaukee, WI (US); Versiti Blood Research Institute, Inc., Milwaukee, WI (US)

(72) Inventors: Qizhen Shi, Brookfield, WI (US); Bonnie Nelson Dittel, Oconomowoc, WI (US)

(21) Appl. No.: 18/870,850

(22) PCT Filed: Jun. 9, 2023
(86) PCT No.: PCT/US2023/068251

§ 371 Date: Dec. 2, 2024

Related U.S. Application Data
(60) Provisional application No. 63/351,088, filed on Jun. 10, 2022.

(51) Int. Cl. A61K 35/28; A61K 31/7088; A61K 35/12; A61K 38/17; A61K 39/00; A61K 40/10; A61K 40/22; A61K 40/24; A61K 40/34; A61K 40/41; A61P 37/06; C12N 15/11

(52) U.S. Cl. CPC A61K 35/28; A61K 31/7088; A61K 38/1774; A61K 39/0008; A61K 40/10; A61K 40/22; A61K 40/24; A61K 40/34; A61K 40/416; A61P 37/06; C12N 15/11; A61K 2035/124; A61K 2039/5154; C12N 2830/34

(57) ABSTRACT

Disclosed herein are methods and compositions for inducing immune tolerance in a subject. The methods comprise expressing the truncated MOG polypeptide described herein in HSC and transplanting HSC in a subject in need to induce immune tolerance.

### STATEMENT REGARDING FEDERALLY SPONSORED RESEARCH

Not Applicable

### SEQUENCE LISTING

A Sequence Listing accompanies this application and is submitted as an XML file of the sequence listing named “650053_00969_sequences.xml” which is 16,726 bytes in size and was created on Jun. 8, 2023. The sequence listing is electronically submitted via Patent Center with the application and is incorporated herein by reference in its entirety.

### FIELD

The field of the invention relates to methods and compositions for antigen specific immune tolerance for auto-immune disease through platelet targeted gene therapy.

### BACKGROUND

Multiple sclerosis (MS) is a chronic inflammatory autoimmune disease associated with gradual degeneration of myelination in the central nervous system (CNS). Currently there is no cure for MS and available treatment requires long-term disease modifying therapy, which is often associated with severe complications. While the exact pathogenic mechanism of MS development is still unclear, evidence suggests that inflammation initiated by antigen-specific T cells leads to disability in patients with MS. Myelin oligodendrocyte glycoprotein (MOG) is expressed by oligodendrocytes and plays a role in the myelination of nerves in the CNS. Demyelination occurs during MS development and contributes to the multitude of symptoms associated with MS pathogenesis. The mouse model of MS, experimental autoimmune encephalomyelitis (EAE), has been widely used for MS studies as they share similar clinical and pathophysiological features. EAE, which is characterized by inflammation leading to demyelination and neuronal damage, can be induced by immunization with the MOG₃₅₋₅₅ peptide. Of note, all FDA approved drugs for MS have shown efficacy in EAE, demonstrating that the EAE model is valuable for evaluating novel interventions for the treatment of MS.

Antigen-specific immune tolerance induction via gene therapy is an attractive approach for the treatment of patients with MS.

### SUMMARY

The present invention is a method of inducing immune tolerance to a protein of interest through the use of a gene therapy approach targeting expression of the protein of interest inside cells of the megakaryocyte lineage, including platelets. The patients who may benefit from this type of immune tolerizing approach include those with allergies, auto-immune disease, transplant recipients and those who have a progressive demyelinating disease such as multiple sclerosis.

In one aspect, the disclosure provides a method of inducing immune tolerance to a protein of interest comprising the steps of: (a) introducing a polynucleotide encoding and capable of expressing the protein of interest, preferably a myelin oligodendrocyte glycoprotein (MOG) polypeptide, in hematopoietic stem cells, and (b) administering the hematopoietic cells of step (a) comprising the polynucleotide into a subject, wherein the subject develops immune tolerance to the protein of interest.

In another aspect, the disclosure provides a method of treating an autoimmune disease in a subject, the method comprising: a) administrating an engineered hematopoietic cell comprising a polynucleotide capable of expressing a protein of interest associated with an autoimmune disease, optionally, a MOG protein, to the subject in an amount effective to induce immune tolerance.

In another aspect, the disclosure provides a composition for inducing immune tolerance to a subject in need, the composition comprising a polynucleotide encoding a protein of interest, optionally a MOG peptide, operably connected to a heterologous promoter, optionally a platelet-specific promoter.

In yet another embodiment, the disclosure provides a method of inducing immune tolerance in a subject comprising administering a therapeutically effective amount of the composition described herein to the subject in need thereof.

In another aspect, the disclosure provides a method to treat degeneration of myelination in the central nervous system, the method comprising the steps of: (a) transducing hematopoietic stem cells with a polynucleotide encoding a MOG polypeptide operably connected to a heterologous promoter, preferably a platelet specific promoter, and (b) administering the transfected cells of step (a) into a subject, wherein the truncated MOG polypeptide is expressed, and wherein the subject develops immune tolerance.

### BRIEF DESCRIPTION OF THE DRAWINGS

FIG. 1A-1B. Generation and evaluation of platelet-specific MOG expression lentiviral vectors. (FIG. 1A) Schematic diagram of MOG_{TD}, MOG₁₋₁₅₇, and MOG_{FL} expression cassettes. Each MOG expression cassette was placed under the control of the platelet-specific αIIb promoter. (FIG. 1B) MOG expression in a promegakaryocyte cell line, Dami cells. Lentiviral vectors harboring the 2bMOG_{TD}, 2bMOG₁₋₁₅₇, or 2bMOG_{FL} expression cassette were produced by transient transfection of HEK293 cells. Dami cells were transduced with lentiviruses. After 72 hours of culture, cells were stained with anti-MOG antibody with or without permeabilization and analyzed by flow cytometry. Representative figures from flow cytometry analysis are shown.

FIG. 2A-2G. Targeting MOG expression to platelets did not affect the leukocyte profile. Sca-1⁺ HSCs/progenitors isolated from CD45.2 WT C57BL/6J donors were transduced with lentivirus and transplanted into CD45.1 recipients preconditioned with 660 cGy total body irradiation. After HSCT and BM reconstitution, blood samples were collected from recipients at various time points, and leukocytes were stained for CD45.1, CD45.2, CD4, CD8, and B220. After staining, cells were analyzed by flow cytometry. Representative results from week 7 from one trial after HSCT are shown. (FIG. 2A) Schematic diagram of experimental design to generate 2bMOG_{TD}, 2bMOG₁₋₁₅₇, 2bMOG_{FL}, and 2bGFP recipients. (FIG. 2B) Representative dot plots from flow cytometry analysis of chimerism. (FIG. 2C) Chimerism in transduced recipients at various time points. (FIG. 2D) The chimerism in transduced recipients. (FIG. 2E) The percentage of CD4 T cells in transduced recipients. (FIG. 2F) The percentage of CD8 T cells in transduced recipients. (FIG. 2G) The percentage of B220 cells in transduced recipients. There were no significant differences in chimerism, CD4, CD8, or B cells between 2bMOG-transduced groups and the 2bGFP control group. (FIG. 2D, 2E, 2F, 2G) Each data point represents one mouse. Three replicate experiments were performed

FIG. 3A-3E. Platelet-MOG expression in 2bMOG-transduced recipients. Blood samples were collected from 2bMOG-transduced recipients after at least 3 weeks of BM reconstitution. Platelets were isolated, stained for CD41 and MOG with or without cell permeabilization, and analyzed by flow cytometry. 2bGFP was used as a control. (FIG. 3A) Representative dot plots from flow cytometry analysis by surface staining of MOG expression 3 weeks after HSCT. (FIG. 3B) The percentages of MOG positive platelets in recipients by surface staining are shown. For individual mice analyzed more than once over the study, the average platelet MOG expression was calculated. (FIG. 3C) Representative dot plots from flow cytometry analysis by intracellular staining of MOG expression 3 weeks after HSCT is shown. (FIG. 3D) The percentages of MOG positive platelets in indicated recipients by intracellular staining is shown. For individual mice analyzed more than once over the study, the average platelet MOG expression was calculated. (FIG. 3E) Representative mean fluorescent intensity (MFI) of intracellular MOG expression in transduced recipients from one trial at 3 weeks after HSCT is shown. MOG MFI was analyzed by flow cytometry analysis through intracellular staining. **P<0.01; ****P<0.0001. “n.s.” indicates no statistically significant difference between the two groups. (FIG. 3B, 3D, 3E) Each data point represents one mouse. Data were summarized from four trials. MFI, mean fluorescence intensity

FIG. 4A-4H. Platelet-specific MOG_{FL}, not MOG_{TD}, expression ameliorated EAE disease severity. After lentivirus transduction of HSCs followed by HSCT and at least 3 months of BM reconstitution, mice were challenged with MOG₃₅₋₅₅ peptide emulsified in CFA along with intraperitoneal injection of pertussis toxin on days 0 and 2. Animals were monitored, and clinical scores were recorded during the study period of day 5-31 after EAE induction. (FIG. 4A) Clinical scores in 2bMOG_{FL}-, 2bMOG_{TD}-, and 2bGFP-transduced recipients through the study period after EAE induction. N=10 in each group. (FIG. 4B) The percentage of paralysis-free transduced recipients after EAE induction during the study period of 5-31 days. Mice with a clinical score ≥2.5 were defined as having paralysis. N=10 in each group. (FIG. 4C) Clinical scores in transduced recipients at day 17 after EAE induction. (FIG. 4D) Cumulative disease scores in transduced recipients during the study period. (FIG. 4E) Surface expression of MOG in transduced recipients. (FIG. 4F) Intracellular expression of MOG in transduced recipients. (FIG. 4G) PCR analysis of MOG proviral DNA in leukocytes from transduced recipients. DNA was purified from peripheral blood leukocytes at 3 weeks after HSCT and the MOG expression cassette was amplified by PCR using primers designed for each construct. WT mouse FVIII was used as an internal control for DNA integrity. (FIG. 4H) Quantitative real-time PCR analysis was used to determine the average copy number of LTR in 2bMOG_{TD}, 2bMOG_{FL}, 2bGFP-transduced recipients. *P<0.05; **P<0.01; ***P<0.001; ****P<0.0001. “n.s.” indicates no statistically significant difference between the two groups. (FIGS. 4C-4F, 4H) Each data point represents one mouse. Data were summarized from two trials.

FIG. 5A-5J. Eliminating MOG transmembrane domains (MOG₁₋₁₅₇) enhanced clinical efficacy in immune tolerance induction in EAE. After lentivirus transduction of HSCs followed by HSCT and at least 3 months of BM reconstitution, EAE was induced. Clinical scores and body weights were monitored daily during the study period of 5-31 days after EAE induction. Loss of bladder control (urinary incontinence) during days 5-20 was assessed by visual observation of wetness on the animal's fur on the caudal abdomen. (FIG. 5A) The average daily EAE score of 2bMOG₁₋₁₅₇-, 2bMOG_{FL}-, 2bGFP-transduced recipients over time are shown. (FIG. 5B) Body weights of 2bMOG₁₋₁₅₇-, 2bMOG_{FL}-, 2bGFP-transduced recipients over time are shown. (FIG. 5C) The percentages of transduced recipients that were paralysis free after EAE induction are shown. (FIG. 5D) The cumulative scores of 2bMOG₁₋₁₅₇, 2bMOG_{FL}, and 2bGFP recipients up to 31 days after EAE induction are shown. (FIG. 5E) The EAE score of 2bMOG₁₋₁₅₇-, 2bMOG_{FL}-, and 2bGFP-transduced recipients at day 17 after EAE induction are shown. (FIG. 5F) Body weights of the 2bMOG₁₋₅₇, 2bMOG_{FL}, and 2bGFP recipients at day 17 after EAE induction are shown. (FIG. 5G) Days of bladder control loss in transduced recipients after EAE induction during the study period are shown. (FIG. 5H) Surface expression of MOG in transduced recipients is shown. (I) Intracellular expression of MOG in transduced recipients is shown. *P<0.05; **P<0.01; ***P<0.001; and ****P<0.0001. “n.s.” indicates no statistically significant difference between the two groups. (FIGS. 5D-5H, 5I) Each data point represents one mouse. Data were summarized from three trials.

FIG. 6A-6H. Platelet-targeted MOG₁₋₁₅₇ expression lead to Treg accumulations and suppressed CD8 T cell recall responses to MOG₃₅₋₅₅ stimulation. Leukocytes from peripheral blood at 11 weeks after transplantation of transduced HSCs before EAE induction and 20 days after EAE induction were stained for CD4, CD25, and Foxp3 and analyzed by flow cytometry. One to two months after EAE induction, splenocytes from transduced recipients were isolated, labeled with Violet CellTracer, and cultured with various doses (0, 2, 10, 50, and 100 mg/ml) of MOG₃₅₋₅₅ peptide for 3 days. Cells were harvested and stained for CD4 and CD8. Zombie Red™ staining was used to exclude dead cells. After staining, cells were analyzed by flow cytometry. (FIG. 6A) Representative dot plots of flow cytometry analysis of Treg cells in transduced recipients after EAE induction are shown. (FIG. 6B) The percentages of Foxp3+ Treg cells in transduced recipients after EAE induction are shown. (FIG. 6C) The percentages of CD25+Foxp3+ Treg cells in transduced recipients after EAE induction are shown. (FIG. 6D) The percentages of Foxp3+ Treg cells in transduced recipients before EAE induction are shown. (FIG. 6E) The percentages of CD25+Foxp3+ Treg cells in transduced recipients before EAE induction are shown. (FIG. 6F) The workflow of the T cell proliferation assay is shown. (FIG. 6G) The stimulation index of CD4 T cell proliferation in each group cultured with various concentrations of MOG₃₅₋₅₅ is shown. (FIG. 6H) The stimulation index of CD8 T cell proliferation in indicated groups with various concentrations of MOG₃₅₋₅₅ is shown. The stimulation index (SI) was calculated as follows: SI=(the percentage of proliferating daughter cells in MOG₃₅₋₅₅-treated wells)/(the percentage of proliferating daughter cells in control wells with 0 mg/ml of MOG₃₅₋₅₅). Two-way ANOVA was used to compare T cell stimulation indexes among groups. *P<0.05; **P<0.01. (FIG. 6B-6E, 6G, 6H) Each data point represents one mouse. Data were summarized from two trials.

FIG. 7A-7B. Flow cytometry analysis of T and B cells in peripheral blood. Leukocytes were isolated from peripheral blood cells and stained with CD4, CD8, and B220. Representative dot plots from the time point of 7 weeks after BMT are shown.

FIG. 8A-SC. The correlation of clinical score and body weight in transduced recipients after EAE induction. After 2bMOG-transduction followed by transplantation and 3 months of bone marrow reconstitution, animals were challenged with MOG₃₅₋₅₅ peptide emulsified in the complete Freund's adjuvant along with the intraperitoneal injection of pertussis toxin to induce the development of EAE. Animals were monitored daily between day 5-20 after EAE induction for clinical scores and the changes of body weights. The correlation between clinical scores and body weights was determined by the Pearson test.

FIG. 9A-9C. The correlation of clinical score and MOG expression in platelets in 2bMOG-transduced recipients after EAE induction. The percentages of MOG positive platelets in transduced recipients were determined by flow cytometry. Data shown were the average platelet-MOG expression from each recipients from at least two time points. After 2bMOG-transduction followed by transplantation and 3 months of bone marrow reconstitution, animals were challenged with MOG₃₅₋₅₅ peptide emulsified in the complete Freund's adjuvant along with the intraperitoneal injection of pertussis toxin to induce the development of EAE. Animals were monitored daily between day 5-31 after EAE induction for clinical scores. The correlation between clinical scores and platelet-MOG expression was determined by the Pearson test. (FIG. 9A) The correlation in all recipients, including 2bMOG₁₋₁₅₇-, 2bMOG_{FL}-, and 2bGFP-transduced recipients. (FIG. 9B) The correlation in 2bMOG₁₋₁₅₇-transduced recipients. (FIG. 9C) The correlation in 2bMOG_{FL}-transduced recipients.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION

One objective of the present invention is to provide a method and composition which induces immune tolerance in subjects to a protein or peptide of interest, and also the use of engineered HSCs to induce immune tolerance. Also provided herein is a method to produce and use the composition to induce immune tolerance in patients. The patients who may benefit from this type of immune-tolerizing approach include those with myasthenia gravis, ankylosing spondylitis, neuromyelitis optica, multiple sclerosis, rheumatoid arthritis, bullous pemphigoid, narcolepsy, Hashimoto thyroiditis, Graves disease dermatitis herpetiformis, or vitiligo.

The present disclosure provides a method of inducing immune tolerance against a protein of interest, specifically, for example, myelin oligodendrocyte glycoprotein (MOG) peptide. The method comprising the steps of (a) introducing a polynucleotide encoding and capable of expressing a myelin oligodendrocyte glycoprotein (MOG) polypeptide specifically inside megakaryocytes and/or platelets into hematopoietic stem cells, and (b) administering the hematopoietic cells of step (a) comprising the polynucleotide into a subject, wherein the subject develops immune tolerance. Specifically, the subject develops immune tolerance to MOG, resulting in a reduction in demyelination of neurons and reduction of diseases in which demyelination is a symptom.

In some embodiments, the protein of interest is associated with an autoimmune disease. For example, the protein of interest can be myelin oligodendrocyte glycoprotein (MOG) polynucleotide. In some aspects, the MOG polynucleotide has been modified to improve its expression from the constructs described herein. For example, the inventors have generated a modified MOG polynucleotide that contains two copies of the MOG peptide from amino acids 64-146 termed MOG_{D} (SEQ ID NO: 12 and SEQ ID NO: 13). A second modified MOG polynucleotide is MOG₁₋₁₅₇, which harbors a truncated MOG protein without the two transmembrane regions but includes the signal peptide and encompasses amino acids 1-157 of the full length protein (SEQ ID NO: 10 and SEQ ID NO: 11). Another version of the modified MOG polynucleotide is MOG_{FL}, which encodes full length MOG protein, including the transmembrane regions and signal peptides (SEQ ID NO: 8 and SEQ ID NO: 9). The transmembrane domains of SEQ ID NO: 8 are encoded in nucleotides 472-528 and nucleotides 538-606. The transmembrane domains of the amino acid sequence of SEQ ID NO: 9 are present at amino acids 158-176 and 180-202. As used herein, a MOG polynucleotide may comprise the full length MOG polynucleotide or a truncated version as in SEQ ID NO: 8, SEQ ID NO: 10 or SEQ ID NO: 12.

In some embodiments immune tolerance is induced. Immune tolerance is the inhibition of an immune response against a particular antigen and state of unresponsiveness of the immune system to an antigen that would otherwise have the capacity to elicit an immune response. The immune system is generally tolerant of self-antigens, so it does not usually attack the body's own cells, tissues, and organs. However, when tolerance is lost, disorders like autoimmune diseases occur, including multiple sclerosis. The present disclosure provides a method if inducing immune tolerance, specifically to a myelin oligodendrite protein polypeptide in a subject, which results in the reduction in demyelination of oligodendrocytes and nerve cells, allowing for the reduction in autoimmune diseases associated with myelin degeneration or demyelination, including multiple sclerosis. Immune tolerance can be characterized by a number of factors in the subject to be treated. One factor is viable engraftment of the transduced stem cells are capable of expressing the protein or peptide. Another characteristic is the presence of HLA markers, the protein of interest or other molecular markers cloned into the vector. The protein of interest could also be detected in circulating platelets, e.g., the detection of MOG peptide in circulating platelets. Successful tolerization may be evaluated by the lack of antibody development to the protein of interest (e.g., MOG) or a reduction in antibody level from prior to transplant. The lack of antibody production by cells from the patient after challenged with the protein of interest (e.g., MOG) could also be an indicator of induced immune tolerance. In some embodiments, immune tolerance may be characterized by an increase in CD4+Foxp3+ regulatory T cells. Immune tolerance may also be characterized by peripheral clonal deletion of antigen-specific CD4 and CD8 effector cells.

Use of the engineered stem cells in a medical procedure to induce immune tolerance to a target protein of interest (e.g., MOG), a sufficient number of hematopoietic stem cells (HSCs) transfected or transduced with a vector containing the protein of interest and capable of expression and a promoter selected to drive platelet-specific expression. In some aspects, HSCs can be collected from the patient. Suitable methods to collect HSCs include a surgical bone marrow aspiration or mobilize the peripheral blood with a cytokine such as granulocyte colony stimulating factor such that HSC's would migrate from the bone marrow into the periphery where they could be harvested by venipuncture. The resulting isolated cells can be purified to enrich the cells for HSCs through positive or negative selection means. One could enrich the mobilized peripheral blood or bone marrow populations by positively selecting cells expressing known stem cell markers such as CD34⁺, C-kit, Thy1.1^{+/lo}, Slamf1/CD150⁺ or others. In some aspects, the isolated cell population is at least 90% positive for one HSC marker, alternatively at least 95% positive, at least 98% positive, at least 99% positive.

A similar approach to transduce and transplant HSC from alternate sources (e.g., donor, stem cell line) can also be used. HSCs harvested from the patient to be treated, a cord blood source, a related donor, or an un-related donor with appropriately matched HLA can be used in the methods described herein.

Methods of introducing the polypeptide into HSCs are known in the art. For example, HSCs can be mixed with the virus (encoding the protein) or construct using transfection, transduction, injection, transformation or other methods known in the art.

Methods of administering the engineered HSCs include, for example, a bone marrow or HSC transplant on patients using conventional methods known to those of skill in medicine. Briefly, a patient can be pre-conditioned using for example and preferably a sub-lethal dose of total body irradiation or chemotherapy such as busulfan supplemented with anti-thymocyte globulin. The patient can then be administered by intravenous infusion the prepared engineered HSCs containing the transgene of the target protein.

By “hematopoietic stem cell (HSC)”, we mean any cell that has the functional ability to repopulate the hematopoietic system and self-renew. There are three main sources of HSC including the bone marrow (BM), peripheral blood (PB), and cord blood (CB). A variety of methods exist to harvest and purify HSC from a patient. In some embodiment, the engineered HSCs capable of expressing the protein of interest can be infused into the patient as a population of cells which contain both engineered HSC and more differentiated hematopoietic cells derived from the engineered HSCs. Alternatively, one can employ a purification or enrichment strategy based on CD marker selection of the HSC prior to introduction of the polynucleotide encoding the protein of interest. Selection methods commonly use antibodies or can use other binding partner proteins which bind the CD marker of interest. Cells are further purified through the use magnetic beads, columns, or other solid surface means of capturing cells of interest.

Methods for treating subjects with the compositions disclosed herein are provided. As used herein, a “subject” may be interchangeable with “patient” or “individual” and means an animal, which may be a human or non-human animal, in need of treatment. In particular embodiments, the subject is a human subject. The subject may have an autoimmune disease, particularly an autoimmune disease associated with demyelination.

A method of inducing immune tolerance is provided. The method comprises (a) introducing a polynucleotide encoding and capable of expressing a protein of interest, for example, myelin oligodendrocyte glycoprotein (MOG) polypeptide, in hematopoietic stem cells, and (b) administering the engineered hematopoietic cells of step (a) comprising the polynucleotide into a subject, wherein the subject develops immune tolerance to the protein of interest.

The polynucleotide can be construct comprising a heterologous promoter and the encoding sequence of the MOG polypeptide described herein.

The term “construct” or “polynucleotide construct” is a polynucleotide which allows the encoded sequence to be replicated and/or expressed in the target cell. A construct may contain an exogenous promoter, operably linked to any one of the polynucleotides described herein. As used herein, a polynucleotide is “operably connected” or “operably linked” when it is placed into a functional relationship with a second polynucleotide sequence. As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the construct is an expression construct, a vector or a viral vector. A vector is any particle used as a vehicle to artificially carry a foreign nucleic sequence, typically DNA into another cell, where it can be replicated and/or expressed. A vector containing foreign DNA is termed recombinant DNA. The four major types of vectors are plasmids, viral vectors, cosmids, and artificial chromosomes. Expression constructs comprise a heterologous promoter and the nucleic acid sequence encoding protein of interest (e.g., MOG) which is capable of expression in the cell in which it is introduced. The expression constructs include vectors which are capable of directing the expression of exogenous genes to which they are operatively linked. Such vectors are referred to herein as “recombinant constructs,” “expression constructs,” “recombinant expression vectors” (or simply, “expression vectors” or “vectors”) and may be used interchangeably. Suitable vectors are known in the art and contain the necessary elements in order for the gene encoded within the vector to be expressed as a protein in the host cell. The term “vector” refers to a nucleic acid molecule capable of transporting another nucleic acid to which it has been linked. One type of vector is a “plasmid”, which refers to a circular double stranded DNA loop into which additional DNA segments may be ligated, specifically exogenous DNA segments encoding the mutant α-gal protein. Another type of vector is a viral vector, wherein additional DNA segments may be ligated into the viral genome. Viral vectors are incorporated into viral particles that are then used to transport the viral polynucleotide encoding the protein of interest into the target cells. Certain vectors are capable of autonomous replication in a host cell into which they are introduced. Other vectors can be integrated into the genome of a host cell upon introduction into the host cell, and thereby are replicated along with the host genome (e.g., lentiviral vectors). Moreover, certain vectors are capable of directing the expression of exogenous genes to which they are operatively linked. In general, vectors of utility in recombinant DNA techniques are often in the form of plasmids. In the present specification “vector” include expression vectors, such as viral vectors (e.g., replication defective retroviruses (including lentiviruses), adenoviruses and adeno-associated viruses (AAV)), which serve equivalent functions.

The vectors are heterogeneous exogenous constructs containing sequences from two or more different sources. Suitable vectors include, but are not limited to, plasmids, expression vectors, lentiviruses (lentiviral vectors), adeno-associated viral vectors (rAAV), among others and includes constructs that are able to express the protein of interest in HSCs. A preferred vector is a lentiviral vector or adeno-associated vector. Suitable methods of making viral particles are known in the art to be able to transform cells in order to express the protein of interest in HSCs described herein.

Heterologous promoters useful in the practice of the present invention include, but are not limited to, constitutive, inducible, temporally-regulated, developmentally regulated, chemically regulated, tissue-preferred, tissue-specific promoters and cell-type specific. The heterologous promoter may be a plant, animal, bacterial, fungal, or synthetic promoter. Suitable promoters are known and described in the art.

In some embodiments, a platelet specific promoter is used to provide expression in platelets differentiated from the engineered HSCs. Suitable platelet specific promoters are known in the art and include, for example, the platelet specific promoter CD41 integrin alphaIIb (aIIb) promoter. Platelet specific promoters are those in which expression is specific to the megakaryocyte and/or megakaryocyte progenitors. In other embodiments, the promoter may be glycoprotein VI promoter, platelet factor 4 (PF4) promoter glycoprotein Ib alpha promoter, glycoprotein 1B beta promoter, glycoprotein IX promoter and other platelet protein promoters.

Within the vector may be an expression cassette. An expression cassette is a distinct component of vector DNA consisting of a gene and regulatory sequence to be expressed by a transfected cell. The modified MOG of SEQ ID NOs: 8, 10 or 12 may be introduced into expression cassettes.

In some embodiments, the vector may contain a signaling peptide. A signaling peptide is also known as a signal sequence, targeting signal, localization signal, localization sequence, transit peptide, leader sequence or leader peptide. A signaling peptide is a short peptide present at the N-terminus, C-terminus, or internally of most newly synthesized proteins that are destined toward the secretory pathway. In some embodiments the signaling peptide is Amino acid 1-29 of the MOG peptide (AA₁₋₂₉): MACLWSFSWPSCFLSLLLLLLLQLSCSYA (SEQ ID NO: 14)

The vector may be for example a lentiviral vector such as the pWPT-2bOVA vector as described in the examples. Alternatively, a AAV vector may be used, as AAV vectors are commonly used for gene therapy.

The present disclosure provides of method of treating a subject with an autoimmune disease. An autoimmune disease is a condition in which the body's immune system mistakes its own healthy tissues as foreign and reacts to it. Suitably, the autoimmune disease may be one associated with myelin degeneration or demyelination, for example, multiple sclerosis. The methods and compositions provided herein may also be used with additional autoimmune diseases including, but are not limited to, myasthenia gravis, ankylosing spondylitis, neuromyelitis optica, rheumatoid arthritis, bullous pemphigoid, narcolepsy, Hashimoto thyroiditis, Graves disease dermatitis herpetiformis, type I diabetes, hemolytic anemia, thrombocytopenic purpura, Goodpasture's syndrome, pemphigus vulgaris, acute rheumatic fever, systemic lupus erythematosus, celiac disease or vitiligo wherein the polynucleotide is specific to each disease.

In some aspects, the present disclosure provides methods to treat degeneration of myelination in the central nervous system, the method comprising the steps of: (a) transducing hematopoietic stem cells with a polynucleotide encoding a MOG polypeptide operably connected to a heterologous promoter, preferably a platelet specific promoter, and (b) administering the transfected cells of step (a) into a subject, wherein the truncated MOG polypeptide is expressed, and wherein the subject develops immune tolerance to MOG. The term degeneration of demyelination describes a loss of myelin with relative preservation of axons. This results from diseases that damage myelin sheaths or the cells that form them. Suitable diseases associated with degeneration of myelination include, for example, multiple sclerosis and the like.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. In some embodiments, the subject is responsive to therapy with the engineered HSC cells disclosed herein, and include use in combination with one or more additional therapeutic agents. The term “treat” further include the reduction in one or more symptom associated with myelin degeneration, for example, reduction or inhibition of loss of motor function, loss of vision in an eye, reduction or inhibition of loss of mobility in an arm or leg, reduction in sense of numbness in legs, reduction or inhibition of one or more of the following symptoms, including, for example, spasms, fatigue, depression, incontinence issues, sexual dysfunction, and walking difficulties.

As used herein the term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment. Suitably the desired effect may be reducing the amount of a multiplicity of different cytokine storm mediating cytokines.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

Unless otherwise specified or indicated by context, the terms “a”, “an”, and “the” mean “one or more.” For example. “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLE

Platelet-specific transmembrane-domain-less MOG expression induces immune tolerance in experimental autoimmune encephalomyelitis

### Introduction

Inducing antigen-specific immune tolerance is desirable in autoimmune diseases. Multiple sclerosis (MS) is a chronic inflammatory autoimmune disease associated with gradual degeneration of myelination in the central nervous system (CNS), resulting in neurological decline with paresis and eventually disability. Currently, there is no cure for MS. Suppression of inflammatory activity is the basic treatment option. This treatment reduces disease progression and clinical relapse but is not curative and requires long-term disease-modifying therapy, which is often associated with severe complications (1-4). While immune reconstitution (IR) therapies utilizing chemotherapies, monoclonal antibodies, or autologous hematopoietic stem cell (HSC) transplantation (HSCT) may induce disease remission and prevent relapses, IR therapies are still limited by morbidity and mortality (5-8). Thus, it is desired to develop new therapeutic approaches that can prevent or reverse MS.

While the exact pathogenic mechanism of MS development is still unclear, evidence suggests that inflammation initiated by antigen-specific T cells leads to disability in patients with MS (9). Myelin oligodendrocyte glycoprotein (MOG) is expressed by oligodendrocytes and plays a role in the myelination of nerves in the CNS. Demyelination occurs during MS development and contributes to the multitude of symptoms associated with MS pathogenesis (10-13). The mouse model of MS, experimental autoimmune encephalomyelitis (EAE), has been widely used for MS studies as they share similar clinical and pathophysiological features (9). EAE, which is characterized by inflammation leading to demyelination and neuronal damage, can be induced by immunization with the MOG₃₅₋₅₅ peptide (9). Antigen-specific immune tolerance induction via gene therapy is an attractive emerging approach for the treatment of patients with MS (14, 15).

Platelets are a unique target for gene therapy to induce immune tolerance due to their innate protein storage and release capacity, expression of immunomodulatory molecules and cytokines, natural turnover with a life-span of approximately 5 days in mice and 10 days in humans, and their ability to interact with various cells of the immune system (16-22). Our previous studies have demonstrated that targeting coagulation factor VIII (FVIII) expression to platelets under the control of the platelet-specific αIIb promoter (2bF8) results in the storage of FVIII in platelet α-granules and that platelet-derived FVIII can effectively induce antigen-specific immune tolerance in hemophilia A mice even with pre-existing anti-FVIII immunity (23-28). When a similar approach was applied to a hemophilia B model, which results from factor FIX (FIX) deficiency, antigen-specific immune tolerance was induced in hemophilia B mice even with anti-FIX immunity (29, 30). Furthermore, targeting non-coagulant protein ovalbumin (OVA) to platelets under the same promoter used for the FVIII and FIX studies also resulted in neoprotein OVA storage in platelet α-granules, leading to antigen-specific immune tolerance (31, 32).

In this study, we evaluated the potential of platelet-specific MOG expression in immune tolerance induction in an EAE model of autoimmune disease. We examined the impact of the cellular location of the neoprotein when MOG expression was targeted to platelets on the efficacy of immune tolerance induction. We showed that lentivirus-mediated platelet-specific MOG gene delivery to HSCs induced immune tolerance in EAE, and a transmembrane-domain-less truncated MOG significantly enhanced the efficacy in immune tolerance induction.

### Results

### MOG Lentiviral Vector Construction and Lentivirus Verification in Dami Cells

Our previous studies indicated that the subcellular location of the neoprotein expressed in platelets might impact immune tolerance induction in platelet-targeted gene therapy (33). MOG is a transmembrane protein expressed on the cell surface of oligodendrocytes that make up the myelin sheath in the CNS (34, 35). In this study, we made three MOG constructs, two of which contain truncated MOG without the transmembrane domain. Specifically, we investigated whether platelet-targeted MOG expression induced immune tolerance and compared the efficacy of immune tolerance induction between MOG proteins with different subcellular locations.

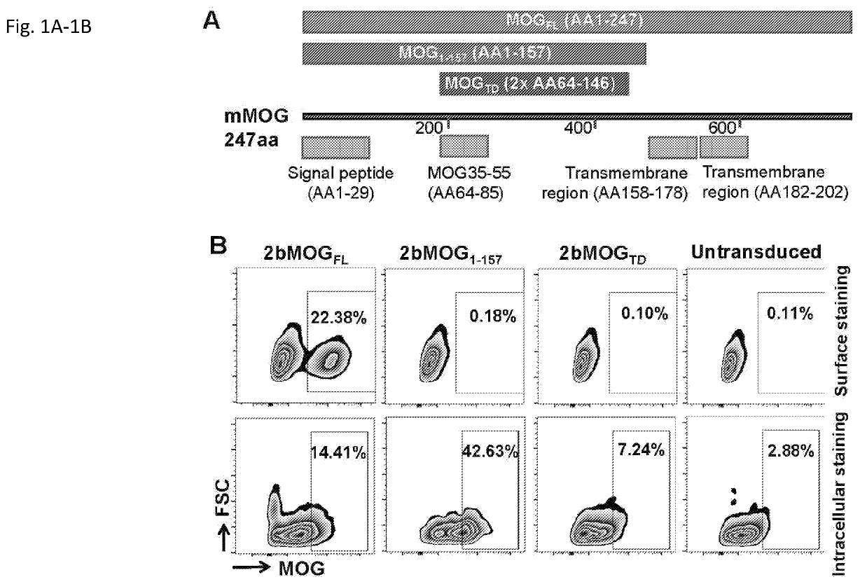

Three expression cassettes (FIG. 1A) were made to introduce MOG protein expression in our study. One was MOG tandem (MOG_{TD}), which contains two copies of the MOG peptide from amino acids 64-146, including the 35-55 encephalitogenic sequence but excluding the two transmembrane regions and the signal peptide. The second one was MOG₁₋₁₅₇, which contains a truncated MOG protein without the two transmembrane regions, but includes the signal peptide from amino acids 1 to 157. The third one was MOG_{FL}, which contains full-length MOG protein from amino acids 1 to 247, including the transmembrane regions and signal peptide.

To validate the viability of each lentiviral vector that harbors a MOG expression cassette under the platelet-specific αIIb promoter, Dami cells, a human promegakaryocyte cell line (36), were transduced with 2bMOG_{TN}, 2bMOG₁₋₁₅₇, or 2bMOG_{FL} lentivirus. MOG protein expression was detected by flow cytometry. As shown in FIG. 1B, using cell surface staining (no permeabilization), MOG neoprotein was detected on Dami cells transduced with the 2bMOG_{FL} lentivirus, but not on the cells transduced with either the 2bMOG₁₋₁₅₇ or 2bMOG_{TD} lentivirus. Of note, when cells were permeabilized followed by MOG intracellular staining, MOG neoprotein was detected in all three groups (FIG. 1B). These data demonstrate that 2bMOG₁₋₁₅₇ and 2bMOG_{TD} can drive MOG intracellular protein expression, but only 2bMOG_{FL} can drive protein expression on the cell surface.

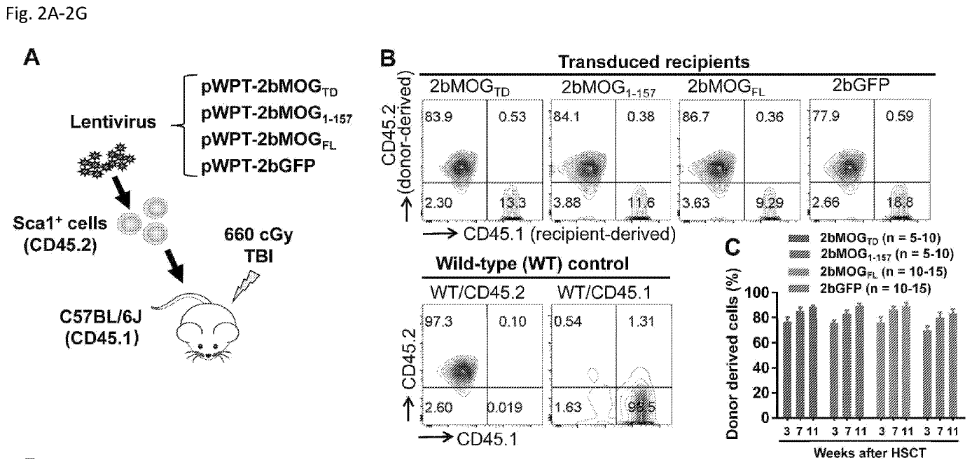

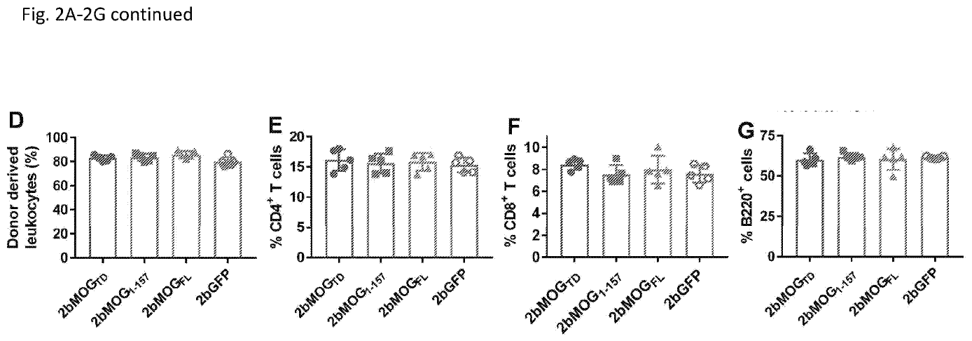

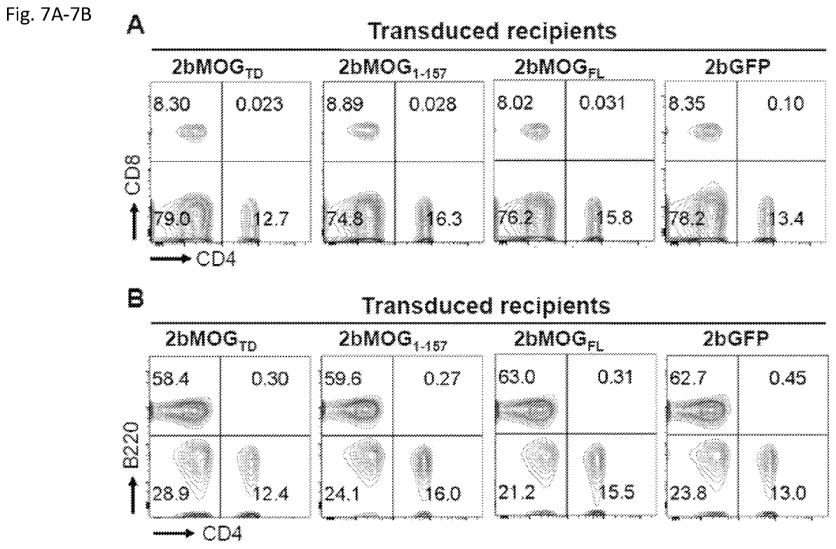

Introducing MOG Expression in Platelets in Mice To introduce MOG expression in platelets, 2bMOG_{TD}, 2bMOG₁₋₁₅₇, and 2bMOG_{FL} lentiviruses were used to transduce Sca-1⁺ bone marrow (BM) cells isolated from WT CD45.2 mice and transplanted into WT CD45.1 recipients that received an optimized non-myeloablative preconditioning regimen for immune tolerance induction in platelet gene therapy (25, 27, 31, 32), 660 cGy total body irradiation (TBI) (FIG. 2A). 2bGFP lentivirus (31) was used as an unrelated control vector in parallel. After BM reconstitution, blood samples were collected for flow cytometry to analyze the engraftment of donor-derived cells (FIG. 2B). Donor-derived leukocytes (CD45.2⁺) gradually increased from 70% at one month to 90% at three months after HSCT (FIG. 2C). There were no significant differences in the engraftment or the percentages of CD4 T cells, CD8 T cells, and B cells among the 2bMOG_{TD}, 2bMOG₁₋₁₅₇, 2bMOG_{FL}, and 2bGFP groups (FIGS. 2D-2G). Representative flow cytometry plots are shown in FIG. 7. These results demonstrate that ectopic expression of MOG to platelets does not influence engraftment or hematopoiesis.

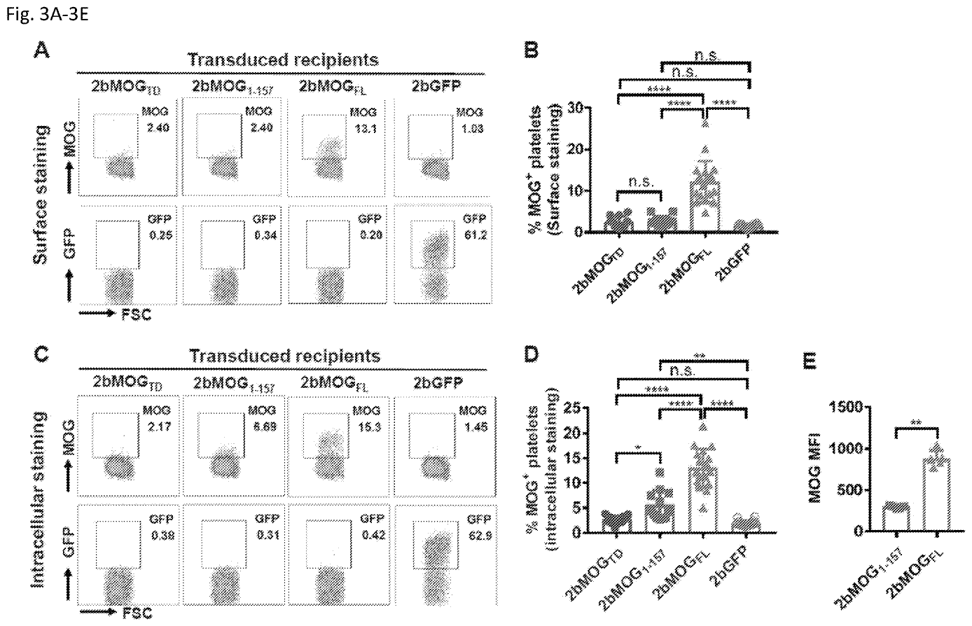

To investigate 2bMOG protein expression and cellular location, platelets from transduced recipients were stained with anti-MOG antibody with or without permeabilization and analyzed by flow cytometry. As shown in FIGS. 3A, B, MOG was detected on 2bMOG-transduced platelets by surface staining in the 2bMOG_{FL} group with an average level of 12.2±5.0% (n=19), which was significantly higher than in the 2bMOG_{TD} and 2bMOG₁₋₁₅₇ groups [2.6±1.4% (n=10) and 2.6±1.2% (n=12), respectively]. There were no significant differences in MOG expression on platelets by surface staining between the 2bMOG_{TD} and 2bMOG₁₋₁₅₇ and the 2bGFP control group (1.5±0.5%, n=20) (FIG. 3B).

When platelets were permeabilized, MOG was detected in 13.0±3.8% of platelets in the 2bMOG_{FL} group and 5.6±3.0% in the 2bMOG₁₋₁₅₇ group, which were significantly higher than in the 2bMOG_{TD} and the 2bGFP groups (2.6±0.9% and 1.8±0.6%, respectively). The MOG positive platelets in the 2bMOG₁₋₁₅₇ group were significantly lower than in the 2bMOG_{FL} group (FIG. 3D). There was no significant difference in MOG expression between the 2bMOG_{TD} and 2bGFP groups (FIGS. 3C, 3D). We further analyzed the mean fluorescence intensity (MFI) of MOG by flow cytometry in 2bMOG₁₋₁₅₇- and 2bMOG_{FL}-transduced recipients. As shown in FIG. 3E, the MFI of MOG expression in the 2bMOG_{FL} group was significantly higher than that in the MOG₁₋₁₅₇ groups (298.4±16.3 vs. 881±99.6, respectively, P<0.01). Together, these data demonstrate that the platelet-specific αIIb promoter-driven MOG fragment expression resulted in differential subcellular localization of MOG protein in platelets. 2bMOG_{FL} lentiviral gene delivery to HSCs resulted in surface expression of MOG by platelets; 2bMOG₁₋₁₅₇ only introduces intracellular expression, and 2bMOG_{TD} failed to drive MOG expression in platelets.

### Platelet-Specific MOG Expression Induced Immune Tolerance to MOG

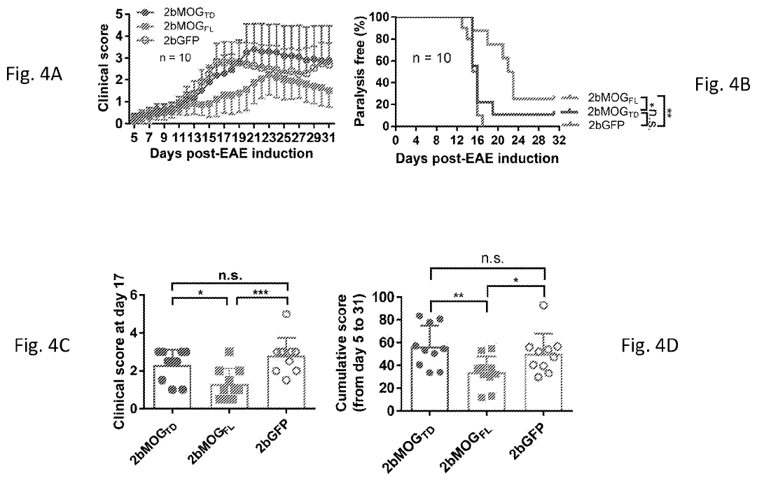

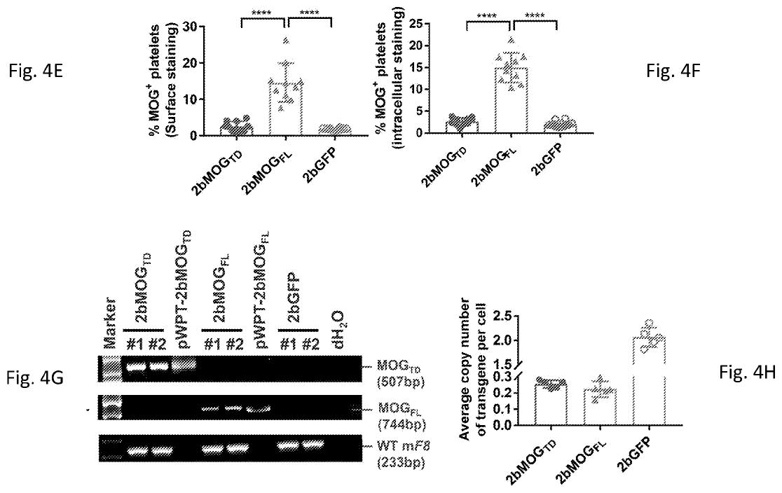

After the BM was fully reconstituted in the recipients, animals were immunized with MOG₃₅₋₅₅ emulsified in complete Freund's adjuvant (CFA) combined with pertussis toxin injections to induce EAE. The clinical score from 5 to 31 days after EAE induction was monitored. As shown in FIG. 4A and Table 1, the daily average clinical score in the 2bMOG_{FL} group was significantly lower than in the 2bMOG_{TD} and 2bGFP groups from days 14 to 20. There was no significant difference in the daily average clinical score between the 2bMOG_{TD} and 2bGFP groups. The percentage of paralysis-free animals in the 2bMOG_{FL} group was significantly higher than in the 2bMOG_{TD} and 2bGFP groups, and there was no statistically significant difference between the 2bMOG_{TD} and 2bGFP groups (FIG. 4B). Seventeen days after EAE induction, the clinical score in the 2bMOG_{FL} group was significantly lower than those in 2bMOG_{TD} and 2bGFP recipients (FIG. 4C). Similarly, the cumulative disease score in the 2bMOG_{FL} group was significantly lower than in the 2bMOG_{TD} and 2bGFP groups, but there was no difference in the day 17 and cumulative disease scores between the 2bMOG_{TD} and 2bGFP groups (FIG. 4C, 4D). These data suggest that platelet-specific MOG_{FL} expression ameliorated EAE disease severity.

TABLE 1
Statistical analysis of the clinical score in 2bMOG_{TD}
and 2bMOG_{FL}-transduced recipients after EAE induction

| Comparisons of the clinical score | Days after EAE induction [symbol] (Individual P-value) | | | | | | | | | | | |
|---|---|---|---|---|---|---|---|---|---|---|---|---|
| in recipients after EAE induction | 14 | 15 | 16 | 17 | 18 | 19 | 20 | 21 | 22 | 29 | 30 | 31 |
| 2bMOG [symbol] vs. 2bMOG [symbol] | ns | [symbol] | [symbol] | [symbol] | [symbol] | [symbol] | [symbol] | [symbol] | [symbol] | [symbol] | ns | [symbol] |
| 2bMOG [symbol] vs. 2bGFP | ns | ns | ns | ns | ns | ns | ns | ns | ns | ns | ns | ns |
| 2bMOG [symbol] vs. 2bGFP | [symbol] | [symbol] | [symbol] | [symbol] | [symbol] | [symbol] | [symbol] | ns | ns | ns | [symbol] | [symbol] |

[symbol]

[symbol]

indicates data missing or illegible when filed

There was no detectable MOG protein expression in the platelets from 2bMOG_{TD}-transduced recipients, regardless of surface- or intracellular-staining (FIGS. 4E, 4F), and platelet-targeted MOG_{TD} expression did not affect the development of EAE (FIGS. 4A-4D) in mice. To examine cell viability in 2bMOG_{TD}-transduced recipients, we used semi-quantitative PCR and quantitative real-time PCR (qPCR) (30) to quantify the MOG proviral DNA. As shown in FIG. 4G, MOG_{TD} and MOG_{FL} proviral DNA were detected by PCR in the 2bMOG_{TD}- and 2bMOG_{FL}-transduced recipients, respectively. The copy number of proviral DNA in the 2bMOG_{TD} and 2bMOG_{FL} groups were comparable (FIG. 4H) when determined by qPCR using primers to amplify the LTR sequence as previously reported (30). These data suggest that the failure of platelet-targeted MOG_{TD} expression in ameliorating EAE is not due to a loss of proviral DNA insertion.

### Eliminating Transmembrane Domains of MOG Expression in Platelets Enhanced the Efficacy in Attenuating Disease Development in EAE

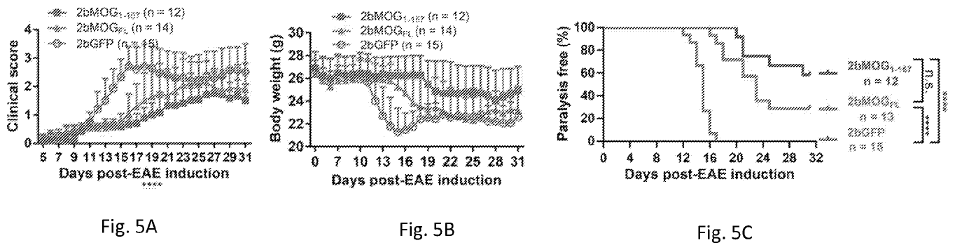

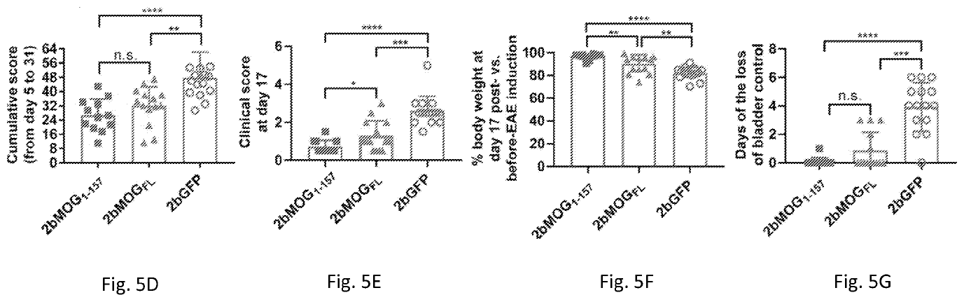

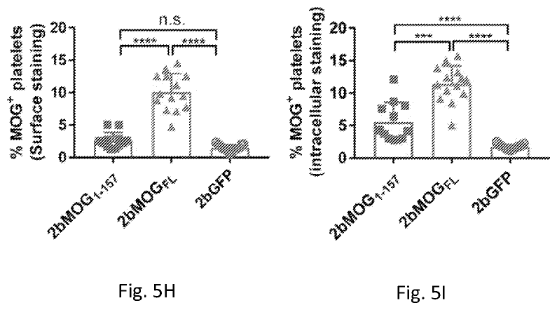

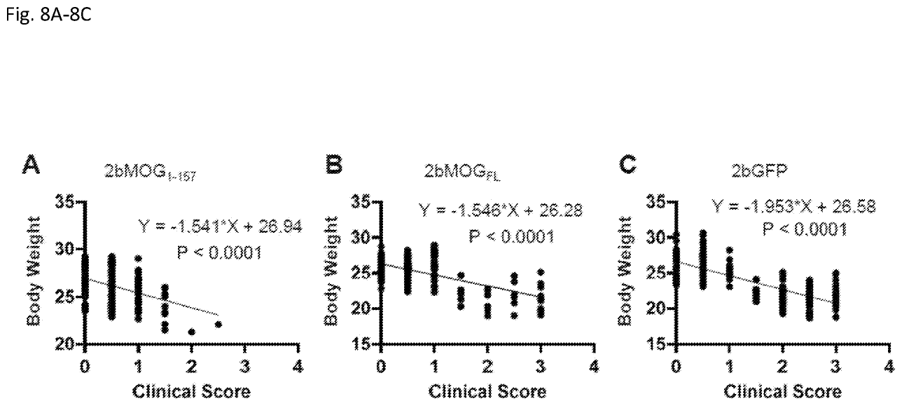

We then compared if the cellular location of MOG protein expression in platelets impacted the efficacy of immune tolerance induction in the EAE model. We compared tolerance induction in 2bMOG₁₋₁₅₇ ad 2bMOG_{FL} recipients. As shown in FIG. 5A and Table 2, clinical scores in both the 2bMOG₁₋₁₅₇ and 2bMOG_{FL} groups were significantly lower than in the 2bGFP control group from day 12 to 19. The clinical score in the 2bMOG₁₋₁₅₇ group was significantly lower than in the 2bMO_{FL} group between days 17-19. Between days 20-31 after EAE induction, clinical scores in the 2bMOG₁₋₁₅₇ group were significantly lower than in the 2bGFP group; however, there were no statistically significant differences between the 2bMOG_{F} and 2bGFP groups although it appears lower in the 2bMOG_{FL} group. The body weights, another parameter for evaluating EAE disease development, in both the 2bMOG₁₋₁₅₇ and the 2bMOG_{FL} groups were significantly higher than in the 2bGFP group between days 12-17 (FIG. 5B and Table 3). Between days 18-31, the body weights in the 2bMOG₁₋₁₅₇ were significantly higher than in the 2bGFP group, but there were no differences between the 2bMOG_{FL} and 2bGFP groups. The body weight in the 2bMOG₁₋₁₅₇ group was significantly higher than the 2bMOG_{FL} group between days 16-26 and 31 (FIG. 5B and Table 3). The clinical scores were negatively correlated with the body weight after EAE induction in all three groups (FIGS. 8A-8C). During the study period, the number of paralysis-free mice after EAE induction in both the 2bMOG₁₋₁₅₇ and 2bMOG_{FL} groups was significantly higher than in the 2bGFP control group. It appears that the number of paralysis-free mice after EAE induction in the 2bMOG₁₋₁₅₇ group was higher than in the 2bMOG_{FL} group, but there was no statistically significant difference between the two groups.

TABLE 2
Statistical analysis of the clinical score in 2bMOG₁₋₁₅₇
and 2bMOG_{FL}-transduced recipients after EAE induction

| Comparisons of the clinical score | Days after EAE induction [symbol] (Individual P-value) | | | | | | | | | | | | | | | | |
|---|---|---|---|---|---|---|---|---|---|---|---|---|---|---|---|---|---|
| in recipients after EAE induction | 12 | 13 | 14 | 15 | 16 | 17 | 18 | 19 | 20 | 21 | 22 | 23 | 24 | 28 | 29 | 30 | 31 |
| 2bMOG [symbol] vs. 2bMOG [symbol] | ns | ns | ns | ns | ns | [symbol] | [symbol] | [symbol] | ns | ns | ns | ns | ns | ns | ns | ns | ns |
| 2bMOG [symbol] vs. 2bGFP | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| 2bMOG [symbol] vs. 2bGFP | [symbol] | [symbol] | [symbol] | [symbol] | [symbol] | [symbol] | [symbol] | [symbol] | ns | ns | ns | ns | ns | ns | ns | ns | ns |

[symbol]

[symbol]

indicates data missing or illegible when filed

TABLE 3
Statistical analysis of the body weight in 2bMOG₁₋₁₅₇ and 2bMOG_{FL}-transduced recipients after EAE induction

| Comparisons of the body weight in | Days after EAE Induction [symbol] (Individual P-value) | | | | | | | | | | | | | | | | | | | |
|---|---|---|---|---|---|---|---|---|---|---|---|---|---|---|---|---|---|---|---|---|
| recipients after EAE induction | 12 | 13 | 14 | 15 | 16 | 17 | 18 | 19 | 20 | 21 | 22 | 23 | 24 | 25 | 26 | 27 | 28 | 29 | 30 | 31 |
| 2bMOG [symbol] vs. 2bMOG [symbol] | ns | ns | ns | ns | [symbol] | [symbol] | [symbol] | [symbol] | [symbol] | [symbol] | [symbol] | [symbol] | [symbol] | [symbol] | [symbol] | ns | ns | ns | ns | [symbol] |
| 2bMOG [symbol] vs. 2bGFP | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| 2bMOG [symbol] vs. 2bGFP | [symbol] | [symbol] | [symbol] | [symbol] | [symbol] | [symbol] | ns | ns | ns | ns | ns | ns | ns | ns | ns | ns | ns | ns | ns | ns |

[symbol]

[symbol]

indicates data missing or illegible when filed

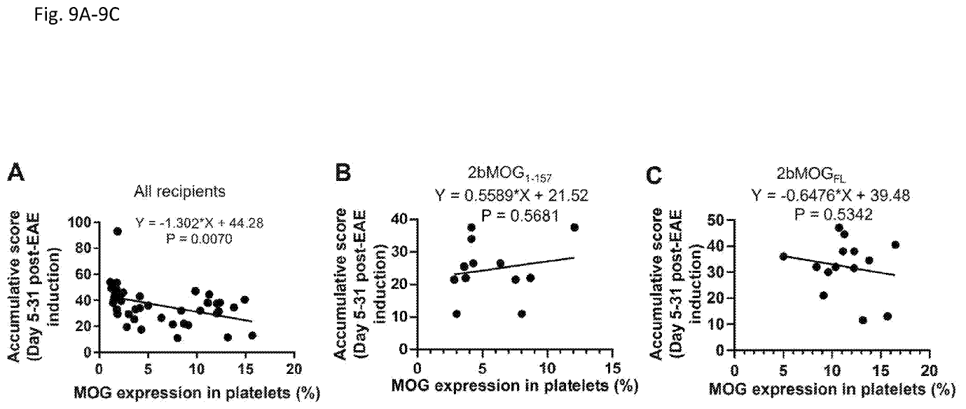

The cumulative disease scores from days 5-31 in the 2bMOG₁₋₁₅₇ and 2bMOG_{FL} groups were 26.7±9.1 (n=12) and 32.1±10.6 (n=14), respectively, which were significantly lower than in the 2bGFP group (47.7±14.5) (n=15) (FIG. 5D). On day 17, when the disease enters the chronic phase (37), clinical scores in the 2bMOG₁₋₁₅₇ and 2bMOG_{FL} groups were significantly lower than in the 2bGFP group (FIG. 5E). Conversely, at day 17, body weights in the 2bMOG₁₋₁₅₇ group were 97.6±3.3% of the base body weight before EAE induction, which was significantly higher than in the 2bMOG_{FL} group (90.3 f 7.9%) and the 2bGFP group (81.9±5.4%) (FIG. 5F). Of note, the cumulative disease score and the score and body weight at day 17 in the 2bMOG₁₋₁₅₇ group were significantly different from those in the 2bMOGa group (FIG. 5D-5F). We also monitored animals to assess loss of bladder control between days 5-20 after EAE induction. As shown in FIG. 5G, only 1 of 12 2bMOG₁₋₁₅₇-transduced recipients had one day of bladder control loss, and 5 of 14 2bMOG_{FL}-transduced recipients had a loss of bladder control varying from 1-3 days. In contrast, 14 of 15 2bGFP-transduced recipients suffered bladder control loss ranging from 2-6 days. The days of bladder control loss in the 2bMOG₁₋₁₅₇ and 2bMOG_{FL} groups was significantly lower than in the 2bGFP group. In the 2bMOG_{FL} group, 10.1±2.8% of platelets expressed MOG protein surface staining and 11.4±2.8% by intracellular staining, but MOG protein expression could only be detected via intracellular staining in the 2bMOG₁₋₁₅₇ group with a percentage of 5.6±3.0 (FIGS. 5H, 5I). The platelet-MOG expression negatively associated with clinical score when GFP control mice were included for comparison (FIG. 9A). However, there were no significant correlations between the percentage of MOG-positive platelets and EAE scores within the groups of 2b MOG₁₋₁₅₇ or MOG₁₋₁₅₇ transduced mice (FIG. 9B, 9C). Collectively, these results demonstrate that both MOG₁₋₁₅₇ and MOG_{FL} gene transfer to platelets induced immune tolerance to MOG with MOG₁₋₁₅₇ exhibiting higher efficacy in ameliorating EAE than MOG_{FL} when MOG expression was targeted to platelets.

MOC₁₋₁₅₇ and MOG_{FL} Induce Immune Tolerance Through Different Mechanisms

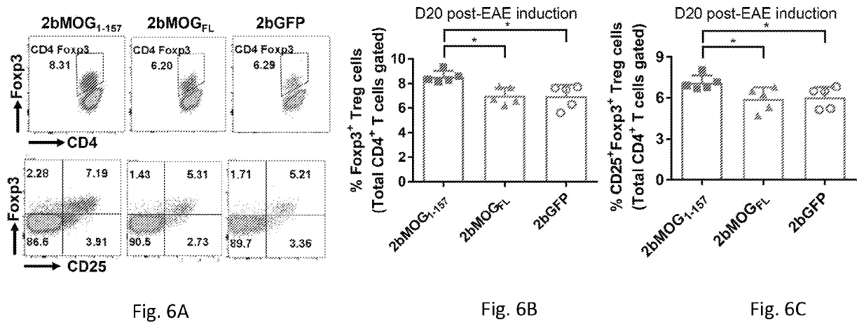

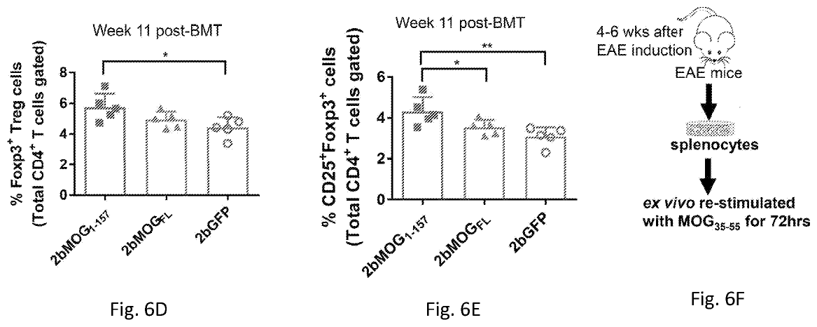

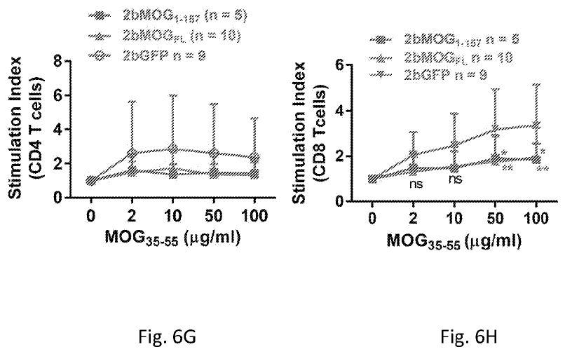

Since CD4⁺Foxp3⁺ T regulatory (Treg) cells are important in suppressing EAE development (37-39), we analyzed Treg cells in peripheral blood by flow cytometry. As shown in FIGS. 6A-6C, on day 20 after EAE induction, Foxp3⁺ and CD25⁺Foxp3⁺ CD4 Treg in the 2bMOG₁₋₁₅₇ group were significantly higher than in the 2bMOG_{FL} and 2bGFP groups, but there was no significant difference between the 2bMOG_{FL} and 2bGFP groups. Before EAE induction, Treg cells in the 2bMOG₁₋₁₅₇ group were significantly higher than in the 2bGFP group, but no statistically significant difference between the 2bMOG₁₋₁₅₇ and 2bMOG_{FL} groups was observed, although there were trends in both the percentage and total number of Treg cells in the 2bMOG_{FL} group higher than the 2bGFP group (FIGS. 6D, 6E). These results indicate that platelet-targeted MOG₁₋₁₅₇ expression can promote Treg cell expansion or induction in 2bMOG₁₋₁₅₇-transduced recipients and that Treg cells further increased after EAE induction, which likely played an important role in suppressing EAE.

To investigate if T cells from 2bMOG-transduced recipients after EAE induction could still respond to MOG restimulation, we performed T cell proliferation assays (FIG. 6F). Splenocytes were isolated from animals at least four weeks after EAE induction and ex vivo co-cultured with various concentrations of MOG₃₅₋₅₅ for 72 hours. CD4 and CD8 T cell proliferation was analyzed by flow cytometry. As shown in FIG. 6G, it appeared that splenic CD4 T cells from the 2bGFP group proliferated more than the 2bMOG₁₋₁₅₇ and 2bMOG_{FL} groups, but there was no statistically significant difference in splenic CD4 T cell proliferation in response to MOG₃₅₋₅₅ restimulation among the 2bMOG₁₋₁₅₇, 2bMOG_{FL}, and 2bGFP groups. Interestingly, the proliferation index of CD8 T cells from the 2bGFP group restimulated with MOG₃₅₋₅₅ peptide in a dose-dependent manner was significantly higher than in the 2bMOG_{FL} and 2bMOG₁₋₁₅₇ groups (FIG. 6H). These results suggested that CD8 T cells were primed in EAE induction and MOG specific CD8 T cells can be induced to tolerize to MOG stimulation when MOG expression was targeted to platelets.

### Discussion

Current therapies for patients with MS mainly target the immune system using immune suppressive agents, which have potential side effects due to systematic immune suppression. Developing antigen-specific immune tolerance is an attractive approach for MS treatment. Several strategies, such as peptide-coupled mononuclear cells or splenocytes, antigen-loaded dendritic cells, or engineered Treg cells have been shown to be efficacious in animal models, yet therapeutics for antigen-specific immune tolerance remains an unmet clinical need for patients with MS (40-44). It has been shown that transient depletion of T cells followed by administration of recombinant myelin could silence relapsing EAE when the treatment was initiated in the early stage, but failed to halt progression (45). Here we described a novel approach to induce immune tolerance in EAE via lentivirus-mediated platelet-specific gene delivery to HSCs to introduce long-term MOG expression in platelets.

Our previous studies have shown that platelet targeted gene transfer can effectively induce antigen-specific immune tolerance in hemophilia A and B mice even with pre-existing immunity (24, 26-30, 46). The present study provides proof-of concept that platelet-targeted gene therapy approach could be applied to induce immune tolerance in autoimmune disease model EAE. Our findings reveal that long-term immune tolerance could be achieved in an EAE model via platelet-targeted MOG expression and that the efficacy of the immune tolerance induction depended on the platelet-MOG expression level and the subcellular location. Full-length MOG protein contains two transmembrane domains and is expressed on the surface of oligodendrocytes (34, 47). The truncated MOG peptide/proteins exhibited different expression levels and subcellular locations depending on the functional domains harbored. We found that when the MOG peptide/protein expression cassette was introduced into the human promegakaryocyte cell line Dami, 2bMOG_{FL} was expressed on the cell surface while 2bMOG_{TD} and 2bMOG₁₋₁₅₇ were expressed intracellularly. Similar expression patterns were found in platelets from the 2bMOG₁₋₁₅₇- and 2bMOG_{FL}-transduced recipients. However, MOG expression was undetectable in 2bMOG_{TD}-transduced platelets, and EAE development was not attenuated in 2bMOG_{TD}-transduced recipients. This might be due to the following: (1) MOG_{TD} is a truncated transgene without a signal peptide sequence, which may result in nonsense-mRNA mediated RNA decay in vivo or (2) platelets are small (compared to Dami cells), thus, the expression of MOG_{TD} did not reach the threshold of being detected by the assay. The undetectable level of MOG expression in 2bMOG_{TD}-transduced platelets might explain the failure to ameliorate EAE in 2bMOG_{TD}-transduced recipients.

In 2bMOG₁₋₁₅₇- and 2bMOG_{TD}-transduced recipients, EAE disease development was attenuated compared to 2bGFP-transduced controls, suggesting that platelet-targeted MOG₁₋₁₅₇ and MOG_{FL} expression can induce immune tolerance to MOG in EAE model mice even though they were primed with MOG together with strong adjuvants (CFA plus pertussis toxin). Although the MOG-positive platelets and MFI of MOG peptides/protein were lower in mice transduced with 2bMOG₁₋₁₅₇ than 2bMOG_{FL}, targeting MOG₁₋₁₅₇ expression to platelets showed better efficacy in attenuating EAE disease development than 2bMOG_{FL}. These data suggest that, to some extent, the efficacy of immune tolerance induction has little to do with the expression level of neoprotein, but that the cellular location in platelets is a critical factor. This conclusion was further evidenced by the findings from our previous platelet-targeted OVA expression study (31). It is known that von Willebrand factor (VWF) propeptide (Vp) can reroute unrelated secretory protein to a storage pathway. In the OVA model study, two lentiviral vectors, which harbored either an OVA expression cassette driven by the αIIb promoter (2bOVA) or Vp-incorporated 2bOVA (2bVpOVA), were used, and the efficacy of immune tolerance induction was compared. While the average platelet-OVA expression levels in 2bVpOVA-transduced recipients were 17-fold lower than in 2bOVA recipients, 2bVpOVA lentiviral gene delivery to HSCs induced OVA-specific immune tolerance was effective as 2bOVA in suppressing anti-OVA antibody production and in preventing skin graft rejection from CAG mice (31). We speculated that, within a certain range of expression levels, the efficacy of immune tolerance induction might not correlate to the expression level of neoprotein, as there are dual mechanisms: peripheral antigen-specific CD4 T cell deletion and Treg cell accumulation (31), that promote immune tolerance in our platelet-targeted gene therapy.

Apart from the expression level of the neoprotein, the subcellular location of the MOG protein was also a major factor in how it impacted immune tolerance induction in platelet-targeted gene therapy. Our previous studies have demonstrated that when FVIII, FIX, or OVA were ectopically targeted to platelets, neoprotein was expressed and stored in platelets, and that platelet-derived neoprotein could effectively induce antigen-specific immune tolerance in treated animals (24, 26, 27, 29, 31). In contrast, when GPIbα or GPIIIa, a platelet membrane protein, was targeted to the surface of the platelet under the same promoter, some of the transduced recipients did produce antibodies against the neoprotein after platelet gene therapy (48, 49). The antibodies in the GPIb model only lasted for three weeks and disappeared subsequently without treatment, indicating immune tolerance could still be established (49). The precise mechanism of why the cellular location of neoprotein impacts immune tolerance induction is still unclear. We speculate that when neoprotein is expressed on the cell surface, it may increase its exposure to the immune system, triggering immune responses if the neoprotein expression level is not sufficient to induce immune tolerance through clonal deletion of antigen-specific CD4 and CD8 T cells. Further studies to understand the underlying mechanisms are warranted. In addition, platelets turnover every 4-5 days in mice, where aged platelets are taken up in the spleen and liver (50). This may lead to more tolerogenic processing of their intracellular contents by splenic/liver macrophages. There is far more inside the platelets than on the membrane, which could be another possibility why neoprotein expressed and stored inside platelets is more tolerogenic than surface expression.

Our previous OVA model studies have demonstrated that long-term antigen-specific immune tolerance is established through dual peripheral tolerance pathways: deletion of peripheral antigen-specific CD4 T cells and induction of antigen-specific Treg cells (31, 32). Using the OVA model, we have shown that the deletion of peripheral antigen-specific CD4 T cells is more prominent in mice with a higher level of platelet-derived OVA expression, whereas with a lower platelet-OVA level but better storage, the increase in antigen-specific Treg cells was dominant (31). In the current study using the MOG model, Treg cells increased in 2bMOG₁₋₁₅₇-transduced recipients but not in 2bMOG_{FL}-transduced recipients. This result partly agrees with the conclusion that expansion of Treg cells dominates in tolerance mechanisms driven by lower levels of platelet-derived neoprotein expression. This could be due to insufficient levels of MOG expressed in 2bMOG₁₋₁₅₇-transduced recipients to induce antigen-specific CD4/CD8 T cell deletion. However, intracellular platelet-MOG might be effective in inducing antigen-specific Treg cells since platelets contain abundant amounts of TGFβ-1, which induce Foxp3 expression (51). Indeed, our current study shows that the percentage of Treg cells in the 2bMOG₁₋₁₅₇ is group was further increased and significantly higher than in the 2bMOG_{FL} and 2bGFP groups.

Besides antigen-specific immune tolerance induced in CD4 T cells after platelet-targeted gene therapy (27, 31), our recent study using the OVA model revealed that OVA-specific effector CD8 T cells can also be deleted in peripheral lymphoid organs after platelet-specific OVA gene transfer (32). In the present study, ex vivo T cell proliferation assays showed that splenic CD8 T cells from the 2bGFP group could proliferate in response to the MOG₃₅₋₅₅ restimulation, but not the 2bMOG₁₋₁₅₇ and 2bMOG_{FL} group, suggesting that the MOG-specific CD8 T cells were tolerized in 2bMOG₁₋₁₅₇- and 2bMOG_{FL}-transduced recipients. This could be a potential mechanism of immune tolerance in 2bMOG-transduced recipients during EAE development. While MS has long been considered a CD4 T cell-mediated disease, accumulating evidence has demonstrated that CD8 T cells also play an important role in the human disease of MS and certain mouse models of EAE (52-54). Thus, inducing immune tolerance via modulating both CD4 and CD8 T cells may be beneficial in controlling EAE/MS disease development. Our current study demonstrates that platelet-targeted MOG expression induced immune tolerance via the Treg and CD8 T cell pathways and that the cellular location of the neoprotein in platelets may govern the tolerization mechanisms. Why the cellular location of neoprotein in our platelet gene therapy results in different clinical efficacy and mechanisms of immune tolerance induction are still unclear. Further studies are needed to illustrate these questions.

In summary, here we evaluated the efficacy of platelet-targeted gene therapy to induce immune tolerance in autoimmune disease model EAE. We found that platelet-targeted MOG₁₋₁₅₇ and MOG_{FL} expression resulted in different subcellular locations of neoprotein and Treg cells responses. CD8⁺ T cells were tolerized after platelet-MOG gene therapy. Both 2bMOG₁₋₁₅₇ and 2bMOG_{FL} expressed in platelets ameliorated EAE, but transmembrane-domain-less MOG₁₋₁₅₇ displayed significantly greater efficacy in inducing immune tolerance and attenuating the development of EAE than full-length MOG_{FL}. Our data demonstrate that targeting transmembrane domain-deleted MOG expression to platelets can effectively induce antigen-specific immune tolerance in EAE. Our study suggests that platelet-targeted gene therapy could be a promising approach for the treatment of patients with autoimmune disease MS.

### Materials and Methods

### Antibodies and Reagents

The following rat anti-mouse monoclonal antibodies (MoAbs) directly conjugated with fluorophore purchased from eBioscience (San Diego, CA, USA) were used in our studies for flow cytometry analysis: CD45.1-FITC, CD45.2-APC eFluor 780, CD4- eFluor 450, CD8-PE Cy7, B220-PerCP Cy5.5, CD25-APC, Foxp3-PE, and the Foxp3 Transcription Factor Staining Buffer Set. Anti-mouse CD42b MoAb conjugated with DyLight-649 was purchased from Emfret Analytics (Eibelstadt, Germany). Rabbit anti-mouse MOG polyclonal antibody was purchased from Aviva System Biology (San Diego, CA, USA). Mouse BD Fc Block was purchased from BD Pharmingen (Franklin Lakes, NJ, USA). BD Cytofix™ fixation buffer was purchased from BD Biosciences (Franklin Lakes, NJ, USA). The EasySep™ Mouse SCA1 Positive Selection Kit was purchased from StemCell Technologies Inc. (Cambridge, MA, USA). The QIAamp DNA Blood Mini Kit was purchased from QIAGEN (Germantown, MD, USA). GoTaq® Green Master Mix was purchased from Promega (Madison, WI, USA). The mouse MOG₃₅₋₅₅ (MEVGWYRSPFSRVVHLYRNGK) (SEQ ID NO: 7) was synthesized by the Protein Chemistry Core laboratory of Versiti Blood Research Institute, Wisconsin, USA. Pertussis toxin was purchased from List Labs (Campbell, CA, USA). CFA was purchased from Chondrex, Inc. (Woodinville, WA, USA).

Tyrode buffer contained 20 mM HEPES, 137 mM NaCl, 13.8 mM NaHCO₃, 0.36 mM NaH₂PO₄, and 2.5 mM KCl. Modified Tyrode buffer was prepared with 5.5 mM glucose, 0.25% BSA, and 1 mM MgCl₂ in Tyrode Buffer. Platelet collection buffer was prepared with modified Tyrode buffer, 3.8% Sodium citrate, and 50 ng/ml Prostaglandin E1. Gey's solution was prepared with 155 mM NH₄Cl and 10 mM KHCO₃. FACS buffer contained 0.5% BSA and 0.01% NaN₃ in DPBS.

### Mice

All the animals used in this study were on the C57BL/6 genetic background. Wild-type (WT) CD45.1 and WT CD45.2 mice used in this study were purchased from Jackson Laboratory (Bar Harbor, ME, USA) and maintained in our animal facility. All mice were kept in pathogen-free microisolator cages in the Biomedical Research Center operated by the Medical College of Wisconsin. Isoflurane or Xylazine/Ketamine was used for anesthesia. Animal studies were performed according to a protocol approved by the Institutional Animal Care and Use Committee of the Medical College of Wisconsin.

### MOG Vector Construction and Lentivirus Production

Genome information for mouse MOG (NM_010814.2) was obtained from the National Center for Biotechnology Information. The MOG_{TD} (2 copies of MOG amino acids 64-146) and MOG_{FL} (full length of MOG protein) cassettes were synthesized by the GeneArt (Thermo Fisher Scientific, Waltham, MA, USA). The MOG₁₋₁₅₇ cassette (amino acid of MOG amino acid 1-157) was amplified by PCR using primers, as shown in Table 4. The pWPT-2bOVA vector (31), which harbors a platelet-specific promoter αIIb, was used as a backbone vector in this study. The MOG_{TD}, MOG₁₋₁₅₇, and MOG_{FL} cassettes were subcloned downstream of the αIIb promoter in the pWPT-2bOVA vector (31) and replaced the OVA cassette to generate the pWPT-2bMOG_{TD}, pWPT-2bMOG₁₋₁₅₇, and pWPT-2bMOG_{FL} vectors, respectively. The recombinant lentiviruses were produced and titrated following protocols described in our previous report (24, 31).

TABLE 4
Sequences and SEQ ID NO of primers used for PCR

| | | SEQ |
|---|---|---|
| | | ID |
| Name | Primer sequence | NO: |
| MOG_{TD} | 5′ ATTCACGCGTGCCATGGAG 3′ | 1 |
| sense | | |
| primer | | |
| MOG_{TD} | 5′ CGGGTCGACTCACGCATCTT 3′ | 2 |
| antisense | | |
| primer | | |
| MOG₁₋₁₅₇ | 5′TTCACGCGTGCCATGGCCTGTTTGTG 3′ | 3 |
| sense | | |
| primer | | |
| MOG₁₋₁₅₇ | 5′ CGGGTCGACTCAAGTCAGCACACCGGGG | 4 |
| antisense | TTGACCCAAT 3′ | |
| primer | | |
| MOG_{FL} | 5 TTCACGCGTGCCATGOCCTGTTTGTG 3′ | 5 |
| sense | | |
| primer | | |
| MOG_{FL} | 5′ CGGGTCGACTCATCAAAAGGGG 3′ | 6 |
| antisense | | |
| primer | | |

### HSCs Transduction and Transplantation

Sca-1⁺ cells were isolated from BM of WT CD45.2 mice using a Mouse SCA1 Positive Selection Kit following the protocol provided by the manufacturer. Sca-1⁺ cells were transduced with lentivirus (pWPT-2bMOG_{TD}, pWPT-2bMOG₁₋₁₅₇, pWPT-2bMOG_{FL} or pWPT-2bGFP lentivirus) following procedures as described in our previous reports (24, 31). After transduction, 1-1.5×10⁶ cells per mouse in 250 μL X-VIVO 10 media were transplanted via retro-orbital venous injection into 6-week-old WT CD45.1 recipients preconditioned with a 660 cGy total body irradiation using a cesium irradiator (Gammacell 40 Exactor, Best Theratronics, Ltd., Ottawa, Canada). Animals were randomly assigned to the groups 2bMOG_{TD}, 2bMOG₁₋₁₅₇, 2bMOG_{FL} or 2bGFP and transplanted with lentivirus-transduced Sca-1⁺ cells, and referenced as 2bMOG_{TD}, 2bMOG₁₋₁₅₇, 2bMOG_{FL}, and 2bGFP recipients, respectively. Blood samples were collected monthly by retro-orbital bleeds with 3.8% sodium citrate anticoagulant (1:10 vol/vol) starting at one month after transplantation, and plasma, leukocytes, and platelets were isolated as previously described (25).

### PCR Detection of Proviral MOG Transgene

For PCR analysis, genomic DNA was purified from peripheral blood leukocytes using QIAamp DNA Blood Mini Kit, and MOG transgene was amplified using GoTaq Green Master Mix. MOG primers were designed to distinguish MOG_{TD} and MOG_{FL} transgenes, and the sequences for primers are listed in Table 4. Mouse F8 (mF8) was used as an internal control to confirm DNA integrity. dH₂O was used a negative control. pWPT-2bMOG_{TD} or pWPT-2bMOG_{FL} plasmid DNA was used as a positive control.

### Flow Cytometry Analysis

Flow cytometry was used to analyze the chimerism of leukocytes and to determine MOG expression in platelets of transduced recipients. For leukocyte chimerism analysis, leukocytes were isolated from peripheral blood after lysing red blood cells with Gey's solution. Cells were resuspended in 50 μL of DPBS containing Fc Block and incubated for 10 minutes to block non-specific binding. Cells were then stained with 100 μL of DPBS containing a combination of multiple fluorophore-conjugated antibodies at 4° C. for 30 minutes. After staining, cells were washed with 1 mL DPBS, resuspended in 200 μL of FACS buffer, and analyzed by an LSRII flow cytometer (BD Bioscience, Sparks, MD, USA). Samples from WT CD45.1 and WT CD45.2 mice were used as controls. Data were analyzed using FlowJo Software (FlowJo, LLC, Ashland, OR, USA). For intracellular staining for Treg cells, after surface staining, the cells were fixed, permeabilized, and stained using the Foxp3 Transcription Factor Staining Buffer Set, following the protocol provided by the manufacturer.

For flow cytometry analysis of platelet MOG expression, platelets were isolated from peripheral blood and stained for MOG protein expression. Briefly, isolated platelets (1-2×10⁶) were fixed with BD Cytofix™ fixation buffer at 4° C. for 30 min and permeabilized with 0.5% Triton-X100 for 20 min on ice. The platelets were centrifuged at 1200 g for 5 min, and the platelet pellet was resuspended in 50 μL of 2% normal goat serum in FACS buffer to block non-specific binding for 30 min. Platelets were then stained with primary rabbit anti-mouse MOG polyclonal antibody (5 μg/ml) in FACS buffer for 30 min and a goat anti-rabbit Alexa Fluor® 568 secondary antibody along with an anti-mouse CD42b antibody directly conjugated with DyLight 649 for 30 min at room temperature. After staining, platelets were washed, resuspended in 200 μL of FACS buffer, and analyzed by flow cytometry.

### Mouse EAE Induction and Disease Assessment

After transplantation and full BM reconstitution, which takes −12 weeks (25), animals were subcutaneously immunized with 200 μg of MOG₃₅₋₅₅ peptide emulsified in CFA along with the intraperitoneal injection of 200 ng of pertussis toxin on days 0 and 2 following the protocol as described in previous reports (55, 56). Animals were monitored daily from day 5-20 after MOG₃₅₋₅₅ immunization to assess whether platelet-specific MOG expression could induce immune tolerance and prevent the development of EAE. Mice receiving 2bGFP-transduced HSCs were used as controls in parallel. The signs of EAE were monitored daily using the EAE scoring system (55-57): 0.5—Partial limp tail; 1—Limp tail; 1.5—Complete limp tail with hind limb ataxia; 2—Limp tail with hind limb paresis; 2.5—Limp tail with one side of hind limb paralysis; 3—Limp tail with both sides of hind limb paralysis; 4—Limp tail, both sides of hind limb paralysis, and forelimb paralysis; and 5—Moribund/death. Besides monitoring the signs of EAE after MOG₃₅₋₅₅ immunization, we also monitored body weight. The loss of bladder control (urinary incontinence) was assessed by visual observation of wetness on the animal's fur on the caudal abdomen.

TABLE 5
The EAE scoring system

| Score | The clinical phenotypes |
|---|---|
| 0 | No disease |
| 0.5 | Partial limp tail |
| 1 | Limp tail |
| 1.5 | Complete limp tail with hind limb ataxia |
| 2 | Limp tail with hind limb paresis |
| 2.5 | Limp tail with one side of hind limb paralysis |
| 3 | Limp tail with both sides of hind limb paralysis |
| 4 | Limp tail, both sides of hind limb paralysis, |
| | and forelimb paralysis |
| 5 | Moribund/death |

### In Vitro T Cell Proliferation Study

One month after EAE induction, splenocytes from transduced recipients were harvested, and red cells were lysed using Red Cell Lysing Buffer. The splenocytes were labeled with CellTrace Violet by CellTrace™ Violet Cell Proliferation Kit. Labeled cells were cultured at 4.5×10⁵ cells/well in flat-bottom 96-well plates with 300 μL of completed RPMI 1640 media containing 2, 10, 50, or 100 μg/ml MOG₃₅₋₅₅. After 72 h culture, the cells were harvested for flow cytometry analysis. Zombie Red™ Fixable Viability Kit staining was used to exclude dead cells. Cells were analyzed by LSRII flow cytometry, and data were analyzed using FlowJo software. T cell proliferation was described by the stimulation index (SI), which was also termed the proliferation index. The stimulation index indicates the fold change in the percentage of proliferating cells after MOG₃₅₋₅₅ stimulation compared to the condition without MOG₃₅₋₅₅ stimulation.

### Statistical Analysis

All data are presented as the mean±SD. All EAE clinical score data were evaluated by the nonparametric Mann-Whitney test for two experimental groups or Kruskal-Wallis test for three groups. The incidences of the loss of bladder control between the two experimental groups were analyzed by Fisher Exact test. The log-rank test was used to determine the difference in paralysis free between the groups. The correlation between clinical scores and body weights was determined by the Pearson test. Statistical comparisons of other data sets were evaluated by the unpaired student t-test for two experimental groups, and the one-way or two-way ANOVA test for three or more groups. Statistical analysis was performed using GraphPad Prism 8 (GraphPad Software, La Jolla, CA). A value of P<0.05 was considered statistically significant.

### REFERENCES

1. Clifford DB, De LA, Simpson DM, Arendt G, Giovannoni G, Nath A. Natalizumab-associated progressive multifocal leukoencephalopathy in patients with multiple sclerosis: lessons from 28 cases. Lancet Neurol (2010) 9:438-46. doi: 10.1016/S1474-4422(10)70028-4
2. Polman C H. O'Connor P W, Havrdova E, Hutchinson M, Kappos L, Miller D H, et al. A randomized, placebo-controlled trial of natalizumab for relapsing multiple sclerosis. N. Engl. J. Med (2006) 354:899-910. doi: 10.1056/NEJMoa044397
3. Calabresi P A, Radue E W, Goodin D, Jeffery D, Rammohan K W, Reder A T, et al. Safety and efficacy of fingolimod in patients with relapsing-remitting multiple sclerosis (FREEDOMS II): a double-blind, randomised, placebo-controlled, phase 3 trial. Lancet Neurol (2014) 13:545-56. doi: 10.1016/S1474-4422(14)70049-3
4. Montalban X, Hauser S L, Kappos L, Arnold D L, Bar-Or A, Comi G, et al. Ocrelizumab versus placebo in primary progressive multiple sclerosis. N. Engl. J. Med (2017) 376:209-20. doi: 10.1056/NEJMoal606468
5. Giovannoni G, Comi G, Cook S, Rammohan K, Rieckmann P, Soelberg S P, et al. A placebo-controlled trial of oral cladribine for relapsing multiple sclerosis. N. Engl. J. Med (2010) 362:416-26. doi: 10.1056/NEJMoa0909494
6. Coles A J, Twyman C L, Arnold D L, Cohen J A, Confavreux C, Fox E J, et al. Alemtuzumab for patients with relapsing multiple sclerosis after disease-modifying therapy: a randomised controlled phase 3 trial. Lancet (2012) 380:1829-39. doi: 10.1016/S0140-6736(12)61768-1
7. Muraro P A, Martin R, Mancardi G L, Nicholas R, Sormani M P, Saccardi R Autologous haematopoietic stem cell transplantation for treatment of multiple sclerosis. Nat. Rev. Neurol (2017) 13:391-405. doi: 10.1038/nrneurol.2017.81
8. Massey J C, Sutton I J, Ma D D F, Moore J J. Regenerating immunotolerance in multiple sclerosis with autologous hematopoietic stem cell transplant. Front Immunol (2018)9:410. doi: 10.3389/fimmu.2018.00410
9. Gold R, Linington C, Lassmann H. Understanding pathogenesis and therapy of multiple sclerosis via animal models: 70 years of merits and culprits in experimental autoimmune encephalomyelitis research. Brain (2006) 129:1953-71. doi: 10.1093/brain/aw1075
10. Khare P, Challa D K, Devanaboyina S C, Velmurugan R, Hughes S, Greenberg B M, et al. Myelin oligodendrocyte glycoprotein-specific antibodies from multiple sclerosis patients exacerbate disease in a humanized mouse model. J. Autoimmun (2018) 86:104-15. doi: 10.1016/j.jaut.2017.09.002
11. Jarius S, Paul F, Aktas O, Asgari N, Dale R C, de S J, et al. MOG encephalomyelitis: international recommendations on diagnosis and antibody testing. J. Neuroinflammation (2018) 15:134. doi: 10.1186/s12974-018-1144-2
12. Weber M S, Derfuss T, Bruck W. Anti-myelin oligodendrocyte glycoprotein antibody-associated central nervous system demyelination-a novel disease entity? JAMA Neurol (2018) 75:909-10. doi: 10.1001/jamaneurol.2018.1055
13. Weber M S, Derfuss T, Metz I, Bruck W. Defining distinct features of anti-MOG antibody associated central nervous system demyelination. Ther. Adv. Neurol. Disord (2018) 11:1756286418762083. doi: 10.1177/1756286418762083
14. Islam M A, Kundu S, Hassan R. Gene therapy approaches in an autoimmune demyelinating disease: Multiple sclerosis. Curr. Gene Ther (2020) 19:376-85. doi: 10.2174/1566523220666200306092556
15. Lutterotti A, Sospedra M, Martin R. Antigen-specific therapies in MS—current concepts and novel approaches. J. Neurol. Sci (2008) 274:18-22. doi: 10.1016/j.jns.2008.05.021
16. Shi Q, Montgomery R R. Platelets as delivery systems for disease treatments. Adv. Drug Deliv. Rev (2010) 62:1196-203. doi: 10.1016/j.addr.2010.06.007
17. Smyth S S, McEver R P, Weyrich A S, Morrell C N, Hoffman M R, Arepally G M, et al. Platelet functions beyond hemostasis. J. Thromb. Haemost (2009) 7:1759-66. doi: 10.1111/j.1538-7836.2009.03586.x
18. Gawaz M, Langer H, May A E. Platelets in inflammation and atherogenesis. J. Clin. Invest (2005) 115:3378-84. doi: 10.1172/JCI27196
19. Elzey B D, Tian J, Jensen R J, Swanson A K, Lees J R, Lentz S R, et al. Platelet-mediated modulation of adaptive immunity. A communication link between innate and adaptive immune compartments. Immunity (2003) 19:9-19. doi: 10.1016/S1074-7613(03)00177-8
20. Semple J W, Italiano J E Jr., Freedman J. Platelets and the immune continuum. Nat. Rev. Immunol (2011) 11:264-74. doi: 10.1038/nri2956
21. Josefsson E C, Vainchenker W, James C. Regulation of platelet production and life span: Role of bcl-xL and potential implications for human platelet diseases. Int. J. Mol. Sci (2020) 21:7591. doi: 10.3390/ijms21207591
22. Lebois M, Josefsson E C. Regulation of platelet lifespan by apoptosis. Platelets (2016) 27:497-504. doi: 10.3109/09537104.2016.1161739
23. Shi Q, Wilcox D A, Fahs S A, Weiler H, Wells C W, Cooley B C, et al. Factor VIII ectopically targeted to platelets is therapeutic in hemophilia a with high-titer inhibitory antibodies. J. Clin. Invest (2006) 116:1974-82. doi: 10.1172/JCI28416
24. Shi Q, Wilcox D A, Fahs S A, Fang J, Johnson B D, D U L M, et al. Lentivirus-mediated platelet-derived factor VIII gene therapy in murine haemophilia a. J. Thromb. Haemost (2007) 5:352-61. doi: 10.1111/j.1538-7836.2007.02346.x
25. Shi Q, Fahs S A, Wilcox D A, Kuether E L, Morateck P A, Mareno N, et al. Syngeneic transplantation of hematopoietic stem cells that are genetically modified to express factor VIII in platelets restores hemostasis to hemophilia a mice with preexisting FVIII immunity. Blood (2008) 112:2713-21. doi: 10.1182/blood-2008-02-138214
26. Schroeder J A, Chen Y, Fang J, Wilcox D A, Shi Q. In vivo enrichment of genetically manipulated platelets corrects the murine hemophilic phenotype and induces immune tolerance even using a low multiplicity of infection. J. Thromb. Haemost (2014) 12:1283-93. doi: 10.1111/jth.12633
27. Chen Y, Luo X, Schroeder J A, Chen J, Baumgartner C K, Hu J, et al. Immune tolerance induced by platelet-targeted factor VIII gene therapy in hemophilia a mice is CD4 T cell mediated. J Thromb Haemost (2017) 15:1994-2004. doi: 10.111/jth.13800
28. Chen J, Schroeder J A, Luo X, Montgomery R R, Shi Q. The impact of GPIbalpha on platelet-targeted FVIII gene therapy in hemophilia a mice with pre-existing anti-FVIII immunity. J. Thromb. Haemost (2019) 17:449-59. doi: 10.1111/jth.14379
29. Chen Y, Schroeder J A, Kuether E L, Zhang G, Shi Q. Platelet gene therapy by lentiviral gene delivery to hematopoietic stem cells restores hemostasis and induces humoral immune tolerance in FIX(null) mice. Mol. Ther (2014) 22:169-77. doi: 10.1038/mt.2013.197
30. Schroeder J A, Chen J, Chen Y, Cai Y, Yu H, Mattson J G, et al. Platelet-targeted hyperfunctional FIX gene therapy for hemophilia b mice even with preexisting anti-FIX immunity. Blood Adv (2021) 5:1224-38. doi: 10.1182/bloodadvances.2020004071
31. Luo X, Chen J. Schroeder J A, Allen K P, Baumgartner C K, Malarkannan S, et al. Platelet gene therapy promotes targeted peripheral tolerance by clonal deletion and induction of antigen-specific regulatory T cells. Front Immunol (2018) 9:1950. doi: 10.3389/fimmu.2018.01950
32. Li J, Chen J, Schroeder J A, Hu J, Williams C B, Shi Q. Platelet gene therapy induces robust immune tolerance even in a primed model via peripheral clonal deletion of antigen-specific T cells. Mol. Ther. Nucleic Acids (2021) 23:719-30. doi: 10.1016/j.omtn.2020.12.026
33. Cai Y, Shi Q. Platelet-targeted FVIII gene therapy restores hemostasis and induces immune tolerance for hemophilia a. Front Immunol (2020) 11:964. doi: 10.3389/fimmu.2020.00964
34. Brunner C, Lassmann H, Waehneldt T V, Matthieu J M, Linington C. Differential ultrastructural localization of myelin basic protein, myelin/oligodendroglial glycoprotein, and 2′,3′-cyclic nucleotide 3′-phosphodiesterase in the CNS of adult rats. J. Neurochem (1989) 52:296-304. doi: 10.1111/j.1471-4159.1989.tb10930.x
35. Pham-Dinh D, Mattei M G, Nussbaum J L, Roussel G, Pontarotti P, Roeckel N, et al. Myelin/oligodendrocyte glycoprotein is a member of a subset of the immunoglobulin superfamily encoded within the major histocompatibility complex. Proc. Natl. Acad. Sci. U.S.A (1993) 90:7990-4. doi: 10.1073/pnas.90.17.7990
36. Greenberg S M. Rosenthal D S, Greeley T A, Tantravahi R, Handin R I. Characterization of a new megakaryocytic cell line: the dami cell. Blood (1988) 72:1968-77. doi: 10.1182/blood.V72.6.1968.1968
37. Grigg J B, Shanmugavadivu A, Regen T, Parkhurst C N, Ahmed A, Joseph A M, et al. Antigen-presenting innate lymphoid cells orchestrate neuroinflammation. Nature (2021) 600:707-12. doi: 10.1038/s41586-021-04136-4
38. Koutrolos M, Berer K, Kawakami N, Wekerle H, Krishnamoorthy G. Treg cells mediate recovery from EAE by controlling effector T cell proliferation and motility in the CNS. Acta Neuropathol. Commun (2014) 2:163. doi: 10.1186/s40478-014-0163-1
39. Othy S, Jairaman A, Dynes J L, Dong T X, Tune C, Yeromin A V, et al. Regulatory T cells suppress Th17 cell Ca(2+) signaling in the spinal cord during murine autoimmune neuroinflammation. Proc. Natl. Acad. Sci. U.S.A (2020) 117:20088-99. doi: 10.1073/pnas.2006895117
40. Smith C E, Miller S D. Multi-peptide coupled-cell tolerance ameliorates ongoing relapsing EAE associated with multiple pathogenic autoreactivities. J. Autoimmun (2006) 27:218-31. doi: 10.1016/j.jaut.2006.12.002
41. Scott D W. Driving CARs to BARs: The winding road to specific regulatory T cells for tolerance. Front Immunol (2021) 12:742719. doi: 10.3389/fimmu.2021.742719
42. Serra P, Santamaria P. Antigen-specific therapeutic approaches for autoimmunity. Nat. Biotechnol (2019) 37:238-51 doi: 10.1038/s41587-019-0015-4.
43. Lutterotti A, Hayward-Koennecke H, Sospedra M, Martin R. Antigen-specific immune tolerance in multiple sclerosis-promising approaches and how to bring them to patients. Front Immunol (2021) 12:640935. doi: 10.3389/fimmu.2021.640935
44. Moorman C D, Sohn S J, Phee H. Emerging therapeutics for immune tolerance: Tolerogenic vaccines, T cell therapy, and IL-2 therapy. Front Immunol (2021) 12:657768. doi: 10.3389/fimmu.2021.657768
45. Pryce G, O'Neill J K, Croxford J L, Amor S, Hankey D J, East E, et al. Autoimmune tolerance eliminates relapses but fails to halt progression in a model of multiple sclerosis. J. Neuroimmunol (2005) 165:41-52. doi: 10.1016/j.jneuroim.2005.04.009
46. Kuether E L, Schroeder J A, Fahs S A, Cooley B C, Chen Y, Montgomery R R, et al. Lentivirus-mediated platelet gene therapy of murine hemophilia a with pre-existing anti-factor VIII immunity. J. Thromb. Haemost (2012) 10:1570-80. doi: 10.1111/j.1538-7836.2012.04791.x
47. Delarasse C, Della G B, Lu C W, Lachapelle F, Gelot A, Rodriguez D, et al. Complex alternative splicing of the myelin oligodendrocyte glycoprotein gene is unique to human and non-human primates. J. Neurochem (2006) 98:1707-17. doi: 10.1111/j.1471-4159.2006.04053.x
48. Fang J, Hodivala-Dilke K, Johnson B D, D U L M, Hynes R O, White G C, et al. Therapeutic expression of the platelet-specific integrin, alphaIIbbeta3, in a murine model for glanzmann thrombasthenia. Blood (2005) 106:2671-9. doi: 10.1182/blood-2004-12-4619
49. Kanaji S, Kuether E L, Fahs S A, Schroeder J A, Ware J, Montgomery R R, et al. Correction of murine Bernard-soulier syndrome by lentivirus-mediated gene therapy. Mol. Ther (2012) 20:625-32. doi: 10.1038/mt.2011.231
50. Li R, Hoffineister K M, Falet H. Glycans and the platelet life cycle. Platelets (2016) 27:505-11. doi: 10.3109/09537104.2016.1171304
51. Haribhai D, Luo X, Chen J, Jia S, Shi L, Schroeder J A, et al. TGF-beta1 along with other platelet contents augments treg cells to suppress anti-FVIII immune responses in hemophilia a mice. Blood Adv (2016) 1:139-51. doi: 10.1182/bloodadvances.2016001453
52. Kaskow B J, Baecher-Allan C. Effector T cells in multiple sclerosis. Cold Spring Harb. Perspect. Med (2018) 8:a029025. doi: 10.1101/cshperspect.a029025
53. Wagner C A, Roque P J, Mileur T R, Liggitt D, Goverman J M. Myelin-specific CD8+ T cells exacerbate brain inflammation in CNS autoimmunity. J. Clin. Invest (2020) 130:203-13. doi: 10.1172/JCI132531
54. Nichols J M, Kummari E, Sherman J, Yang E J, Dhital S, Gilfeather C, et al. CBD suppression of EAE is correlated with early inhibition of splenic IFN-gamma+CD8+ T cells and modest inhibition of neuroinflammation. J. Neuroimmune. Pharmacol (2021) 16:346-62. doi: 10.1007/s11481-020-09917-8
55. Ray A, Basu S, Miller N M, Chan A M, Dittel B N. An increase in tolerogenic dendritic cell and natural regulatory T cell numbers during experimental autoimmune encephalomyelitis in rras−/− mice results in attenuated disease. J. Immunol (2014) 192:5109-17.
56. Zhang H, Ray A, Miller N M, Hartwig D, Pritchard K A, Dittel B N. Inhibition of myeloperoxidase at the peak of experimental autoimmune encephalomyelitis restores blood-brain barrier integrity and ameliorates disease severity. J. Neurochem (2016) 136:826-36. doi: 10.1111/jnc.13426
57. Kieseier B C, Kiefer R, Clements J M, Miller K, Wells G M, Schweitzer T, et al. Matrix metalloproteinase-9 and -7 are regulated in experimental autoimmune encephalomyelitis. Brain (1998) 121(Pt 1):159-66. doi: 10.1093/brain/121.1.159

### ADDITIONAL EMBODIMENTS

The following disclosure can also be described with reference to the following additional embodiments.

Embodiment 1. A method of inducing immune tolerance to a protein of interest comprising the steps of: (a) introducing a polynucleotide encoding and capable of expressing the protein of interest, preferably a myelin oligodendrocyte glycoprotein (MOG) polypeptide, in hematopoietic stem cells, and (b) administering the hematopoietic cells of step (a) comprising the polynucleotide into a subject, wherein the subject develops immune tolerance to the protein of interest.

Embodiment 2. The method of embodiment 1, wherein the protein of interest is MOG, and wherein the polynucleotide encodes a (i) MOG protein, (ii) truncated MOG protein lacking the transmembrane domain, or (c) two or more peptides comprising a portion of the MOG protein.

Embodiment 3. The method of embodiment 1 or 2, wherein the polynucleotide encodes amino acids 1-157 comprising SEQ ID NO:10 or SEQ ID NO: 8.

Embodiment 4. The method of embodiment 1, wherein the polynucleotide is a construct comprising a heterologous promoter and the encoding sequence of the MOG polypeptide.

Embodiment 5. The method of embodiment 4, wherein the construct comprises a platelet promoter.

Embodiment 6. The method of embodiment 5, wherein the platelet promoter is αIIb promoter.

Embodiment 7. The method of any one of the preceding embodiments wherein the polynucleotide is delivered by contacting the cell with a viral vector comprising the polynucleotide.

Embodiment 8. The method of any one of the preceding embodiments, wherein the gene of step (a) is a MOG peptide that is only expressed intracellularly.

Embodiment 9. The method of any one of the preceding embodiments, wherein the administering step of (b) is a bone marrow transplant or an intravenous infusion of hematopoietic stem cells.

Embodiment 10. The method of any one of the preceding embodiments, wherein the subject develops immune tolerance.

Embodiment 11. The method of embodiment 10, wherein the immune tolerance comprises increased CD4+Foxp3+ cells.

Embodiment 12. The method of any one of the preceding embodiments, wherein the subject has an autoimmune disease, optionally wherein the autoimmune disease is selected from the group consisting of myasthenia gravis, ankylosing spondylitis, neuromyelitis optica, multiple sclerosis, rheumatoid arthritis, bullous pemphigoid, narcolepsy, Hashimoto thyroiditis, Graves disease dermatitis herpetiformis, or vitiligo.

Embodiment 13. The method of embodiment 12, wherein the autoimmune disease is multiple sclerosis.

Embodiment 14. A method of treating an autoimmune disease in a subject, the method comprising: a) administrating an engineered hematopoietic cell comprising a polynucleotide capable of expressing a protein of interest associated with an autoimmune disease, optionally, a MOG protein, to the subject in an amount effective to induce immune tolerance.

Embodiment 15. The method of embodiment 14, where the administrating comprises a bone marrow transplant or an intravenous infusion of the engineered hematopoietic stem cells.

Embodiment 16. The method of embodiment 14 or 15, wherein the method induces immune tolerance in the subject, the immune tolerance increased CD4+Foxp3+ cells in the subject.

Embodiment 17. The method of any one of embodiments 14-16, wherein the autoimmune disease is multiple sclerosis.

Embodiment 18. A composition for inducing immune tolerance to a subject in need, the composition comprising a polynucleotide encoding a protein of interest, optionally a MOG peptide, operably connected to a heterologous promoter, optionally a platelet-specific promoter.

Embodiment 19. The composition of embodiment 18, wherein the protein of interest is a MOG peptide comprises a truncated MOG peptide that does not comprise a transmembrane domain.

Embodiment 20. The composition of embodiment 18 or 19, wherein the polynucleotide encodes the truncated MOG protein of SEQ ID NO:10.

Embodiment 21. The composition of any one of embodiments 18-20, wherein the platelet specific promoter comprises the αIIb promoter.

Embodiment 22. The composition of any one of embodiments 18-21, wherein the polynucleotide expresses the truncated MOG peptide intracellularly.

Embodiment 23. The composition of any one of embodiments 18-22, wherein the composition comprises a viral vector comprising the polynucleotide.

Embodiment 24. A method of inducing immune tolerance in a subject comprising administering a therapeutically effective amount of the composition of any one of embodiments 17-22 to the subject in need thereof.

Embodiment 25. A method to treat degeneration of myelination in the central nervous system, the method comprising the steps of: (a) transducing hematopoietic stem cells with a polynucleotide encoding a MOG polypeptide operably connected to a heterologous promoter, preferably a platelet specific promoter, and (b) administering the transfected cells of step (a) into a subject, wherein the truncated MOG polypeptide is expressed, and wherein the subject develops immune tolerance.

Embodiment 26. The method of embodiment 25, wherein the truncated MOG polypeptide does not contain a transmembrane domain.

Embodiment 27. The method of embodiment 25, wherein the MOG polypeptide is SEQ ID NO:11.

Embodiment 28. The method of any one of embodiments 25-27, wherein the platelet specific promoter is the αIIb promoter.

Embodiment 29. The method of any one of embodiments 25-28, wherein the administering step of (b) is a bone marrow transplant or an intravenous infusion of hematopoietic stem cells.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element In the or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification

1. A method of inducing immune tolerance to a protein of interest in a subject in need thereof, comprising the steps of:
(a) introducing a polynucleotide encoding and capable of expressing the protein of interest, in hematopoietic stem cells, and
(b) administering the hematopoietic cells of step (a) comprising the polynucleotide into the subject,
wherein the subject develops immune tolerance to the protein of interest and wherein the protein of interest comprises a myelin oligodendrocyte glycoprotein (MOG) polypeptide.

2. The method of claim 1, wherein the polynucleotide encodes a (i) MOG protein, (ii) truncated MOG protein lacking the transmembrane domain, or (c) two or more peptides comprising a portion of the MOG protein.

3. The method of claim 1, wherein the polynucleotide encodes SEQ ID NO: 11 or SEQ ID NO: 9.

4. The method of claim 1, wherein the polynucleotide comprises a heterologous promoter operably linked to the encoding sequence of the MOG polypeptide.

5. The method of claim 4, wherein the heterologous promoter comprises a platelet promoter.

6. The method of claim 5, wherein the platelet promoter is αIIb promoter.

7.-10. (canceled)

11. The method of claim 1, wherein the immune tolerance comprises increased CD4+Foxp3+ cells.

12. The method of claim 1, wherein the subject has an autoimmune disease selected from the group consisting of myasthenia gravis, ankylosing spondylitis, neuromyelitis optica, multiple sclerosis, rheumatoid arthritis, bullous pemphigoid, narcolepsy, Hashimoto thyroiditis, Graves disease dermatitis herpetiformis, or vitiligo.

13. (canceled)

14. A method of treating an autoimmune disease in a subject in need thereof, the method comprising:
a) administrating an engineered hematopoietic cell comprising a polynucleotide capable of expressing a protein of interest to the subject in an amount effective to induce immune tolerance, wherein the protein of interest comprises a MOG polypeptide.

15. (canceled)

16. The method of claim 14, wherein the method induces increased CD4+Foxp3+ cells in the subject.

17. (canceled)

18. A composition for inducing immune tolerance to a subject in need thereof, the composition comprising a polynucleotide encoding a protein of interest operably connected to a heterologous promoter, wherein the protein of interest comprises a MOG polypeptide.

19. The composition of claim 18, wherein the MOG polypeptide comprises a truncated MOG peptide that does not comprise a transmembrane domain.

20. The composition of claim 18, wherein the polynucleotide encodes the truncated MOG protein of SEQ ID NO:11.

21. The composition of claim 18, wherein the heterologous promoter comprises a platelet specific promoter.

22. The composition of claim 21, wherein the platelet specific promoter comprises the αIIb promoter.

23. (canceled)

24. A method of inducing immune tolerance in a subject in need thereof comprising administering a therapeutically effective amount of the composition of claim 18 to the subject in need thereof.

25. A method to treat degeneration of myelination in the central nervous system, the method comprising the steps of:
(a) transducing hematopoietic stem cells with a polynucleotide encoding a MOG polypeptide operably connected to a heterologous promoter, and
(b) administering the transfected cells of step (a) into a subject,
wherein the truncated MOG polypeptide is expressed, and wherein the subject develops immune tolerance.

26. The method of claim 25, wherein the heterologous promoter comprises a platelet specific promoter.

27. The method of claim 25, wherein the MOG polypeptide is SEQ ID NO:11.

28. The method of claim 26, wherein the platelet specific promoter is the αIIb promoter.

29. (canceled)
